(19) United States
(12) Patent Application Publication
(10) Pub. No.: US 2023/0233684 A1
(43) Pub. Date: Jul. 27, 2023

(54) USE OF CHELATORS FOR THE PREVENTION OF VISIBLE PARTICLE FORMATION IN PARENTERAL PROTEIN SOLUTIONS

(71) Applicant: Hoffmann-La Roche Inc., Little Falls, NJ (US)

(72) Inventors: Kathrin GREGORITZA, Basel (CH); Andrea ALLMENDINGER, Grenzach-Wyhlen (DE); Satya Krishna Kishore RAVURI, Pfeffingen (CH)

(73) Assignee: Hoffmann-La Roche Inc., Little Falls, NJ (US)

(21) Appl. No.: 18/054,066

(22) Filed: Nov. 9, 2022

Related U.S. Application Data
(63) Continuation of application No. PCT/EP2021/063040, filed on May 18, 2021.

(30) Foreign Application Priority Data
May 19, 2020 (EP) 20175454.6

(51) Int. Cl. A61K 47/18; A61K 47/26; A61K 9/08; A61K 39/395

(52) U.S. Cl. CPC A61K 47/183; A61K 47/26; A61K 9/08; A61K 39/39591

(57) ABSTRACT

The present invention provides methods to prevent the formation of visible particles in aqueous protein formulations, in particular the use of certain chelators, as well as compositions and pharmaceutical products obtained with said method.

### CROSS REFERENCE TO RELATED APPLICATIONS

This application is a continuation of International Application No. PCT/EP2021/063040, filed May 18, 2021, which claims priority to European Patent Application No. 20175454.6, filed May 19, 2020, which is incorporated herein by reference in its entirety.

The present invention relates to the field of aqueous protein compositions, in particular pharmaceutical antibody formulations for parenteral application, which are stabilized against the formation of visible particles.

### BACKGROUND OF THE INVENTION

Formation of visible particles during shelf-life is one of the major concerns associated with biopharmaceutical drug products for parenteral use. Although the full extent of clinical consequences is still unclear, the presence of particles is generally considered as a potential safety risk for patients and therefore, is one of the most common reasons for recall events of parenteral products. (Doessegger et al. 2012)

One of the most common root causes for particle formation in biopharmaceutical formulations is the degradation of polysorbate (PS), such as PS20, which is commonly added to a formulation to protect the protein against interfacial stress.

Polysorbate can be described as a heterogeneous mixture of partial esters of fatty acids with ethoxylated sorbitol or isosorbitol. (Hewitt et al. 2008; Lippold et al. 2017; Kishore et al. 2011b)

Polysorbate degradation by oxidation, as well as chemical or enzymatic hydrolysis is well known and has thoroughly been investigated. The latter mechanism has been reported to be mainly driven by host cell proteins, such as the lysosomal phospholipase A2 (LPLA2), which are co-purified with the protein of interest and can catalyze cleavage of the ester bond in polysorbate. (Labrenz 2014; Dixit et al. 2016)

It was recently reported that these enzymes can possess different specificities against mono-ester or higher-order species of polysorbate resulting in different PS degradation patterns (Graf et al. 2020; Hall et al. 2016). Hydrolytic degradation of PS20 does not only result in a loss of surfactant functionality (Kishore et al. 2011a) but furthermore leads to the release of free fatty acids (FFA), such as lauric or myristic acid, which are sparingly soluble in aqueous solutions and can form visible or sub-visible particles when the FFA concentrations exceed the solubility limits. The solubility of FFAs in solutions is dependent on a variety of different factors, including temperature, pH or the concentration of residual intact polysorbate (Doshi et al. 2015). Trace levels of metal ions like Al³⁺ have been previously shown to interact with FFAs resulting from hydrolytic PS degradation, leading to FFA-metal complexes which eventually precipitate out of aqueous formulations and act as a nucleation seed for visible particles (Allmendinger et al. 2021).

However, the occurrence of FFA particle formation in biopharmaceutical products is often not predictable which leads to the assumption that other nucleation factors might be involved in particle formation. There remains thus a need to provide solutions against the formation of visible particles in parenteral, aqueous protein formulations such as, for example, aqueous preparations (or compositions) of antibodies.

The present invention provides solutions for this problem. More particularly, the present invention provides mitigation options for FFA particle formation below their solubility limit by the addition of excipients (chelators), which can complex multivalent cations and prevent their interaction with fatty acids resulting from polysorbate degradation.

Chelators, such as EDTA or DTPA have been commonly used in biopharmaceutical formulations to prevent oxidative degradation of proteins or polysorbates (Yarbrough et al. 2019; Doyle Drbohlav et al. 2019; Kranz et al. 2019; Doshi et al. 2021; Gopalrathnam et al. 2018). Oxidation can be promoted by the presence of transition metals which can either derive from stainless steel manufacturing equipment (Zhou et al. 2011) or can be introduced through raw materials, e.g. Histidine (European Directorate for the Quality of Medicines).

### SUMMARY OF THE INVENTION

In one embodiment the present invention provides a stable aqueous composition comprising a protein together with pharmaceutically acceptable excipients such as, for example, buffers, stabilizers including antioxidants, and further comprising at least one chelator.

In one embodiment, the present invention provides the use of chelators to prevent the formation of visible particles in aqueous protein formulations.

In one embodiment, the present invention provides the use of chelators in aqueous protein formulations to prevent the formation of visible particles comprising free fatty acids in concentrations below their solubility level.

In another embodiment, the present invention provides a pharmaceutical dosage form comprising a preparation as defined herein, for example an aqueous antibody composition, in a container or vial.

### BRIEF DESCRIPTION OF THE FIGURES

FIG. 1: Hydrodynamic radius (rH) of lauric acid salt particles over time as a function of Al concentration.

FIG. 2: A) DLS intensity and B) inflection points of sigmoidal fits of lauric acid salt particles over time as a function of Al concentration.

FIG. 3: A) Hydrodynamic particles size and B) scattering intensity of lauric acid salt particles over time as a function of metal cation type and concentration.

FIG. 4: Hydrodynamic particles radius of lauric acid salt particles in presence of (A) EDTA and (B) DTPA (C) GLDA and (D) PDTA over time as a function of chelator to aluminum ratio.

FIG. 5: Scattering intensity of lauric acid salt particles in presence of (A) EDTA and (B) DTPA (C) GLDA and (D) PDTA over time as a function of chelator to aluminum ratio.

FIG. 6: (A) Scattering intensity and (B) hydrodynamic particles radius of lauric acid salt particles over time as a function of DTPA to Fe ratio.

FIG. 7: (A) Scattering intensity and (B) hydrodynamic particles radius of lauric acid salt particles over time as a function of DTPA to Al ratio.

### DETAILED DESCRIPTION OF THE INVENTION

Formation of visible particles consisting of free fatty acids (FFA) as a result of surfactant degradation, especially from polysorbate (PS20 and/or PS80) degradation, represents a major challenge in the biopharmaceutical industry as there is limited choice for surfactants in parenteral protein formulations such as, for examples, parenteral preparations of therapeutic antibodies. Reducing or even eliminating polysorbate degradation, and thus the release of FFA, by various means is key as FFA can precipitate to form visible particles, which in turn may impact the quality of a parenteral pharmaceutical product.

Commercially available polysorbates (PS20 and 80) are chemically diverse mixtures containing mainly sorbitan POE fatty acid esters. The main species of PS80 contains a sorbitan head group with 4 chains of polyoxyethylene (POE) extending from it. Theoretically, there are a total of twenty POE units which are attached to each head group, although in practice, there may end up being more or less. Typically, there is a Gaussian-like distribution in the number of POE units, resulting in a heterogeneous mixture. Of the four POE groups attached to the sorbitan head groups, 1 to 3 of them are esterified to fatty acids (FAs) at their ends which can also terminate in a primary alcohol. The FAs found in PS80 are 14 to 18 carbons long and can have up to 3 double bonds along the chain. The most abundant FA is oleic acid (≥58%, 18 carbons, 1 double bond), followed by linoleic (18%, 18 carbons, 2 double bonds). The number of FA substitutions on an individual sorbitan head group can range from zero to 4. PS80 also has isosorbide head groups with zero to 2 FA substitutions. There also exists a significant amount of POE-FAs unattached to the head groups. All of these components result in a diverse heterogeneous mixture which can vary extensively between manufacturers. (Journal of Pharmaceutical Sciences 109 (2020) 633-639). Other fatty acids present, for example, in PS20 include caproic acid, caprylic acid, capric acid, lauric acid, myristic acid, palmitic acid, stearic acid.

PS20 and 80 are available in different grades. In accordance with the present invention, the following grades were tested:
High purity (HP) PS20
Super refined (SR) PS20
High purity (HP) PS80
Super refined (SR) PS80
Pure oleic acid (POA) PS80

All grades were purchased from the specialty chemicals company Croda, headquartered in Snaith, England (herein “Croda”). Compared to the HP PS20 and HP PS80 synthesis process, the SR grades are further purified by the proprietary flash chromatographic process which can remove additional polar and oxidative impurities, such as aldehydres and peroxides from the PS raw material (Doshi et al. 2020a). The pure oleic acid grade was recently introduced by the Chinese Pharmacopoeia (ChP) commission specifying the content of oleate esters to be ≥98.0% for injectable products. Although this high oleic acid grade is no longer mandatory for the use of parenteral products it has recently gained increasing polularity due to its lower propensity to form sub-visible and visible particles upon hydrolytic degradation compared to HP/SR PS20 and HP/SR PS80 (Doshi et al. 2021).

TABLE 1a
US/European/Chinese (Ch) Pharmacopoeia
specification for PS20 and PS80

| | PS20 | PS80 | PS80 High |
|---|---|---|---|
| | (USP/EP/ChP) | (USP/EP/ChP) | oleic acid |
| Fatty acid Ester | (%) | (%) | (ChP) (%) |
| Caproic (C6) | ≤1.0 | n/a | n/a |
| Caprylic (C8) | ≤10.0 | n/a | n/a |
| Capric (C10) | ≤10.0 | n/a | n/a |
| Lauric (C12) | 40.0-60.0 | n/a | n/a |
| Myristic (C14) | 14.0-25.0 | ≤5.0 | ≤0.5 |
| Palmitic (C16) | 7.0-15.0 | ≤16.0 | ≤0.5 |
| Palmitoleic (C16:1) | n/a | ≤8.0 | ≤0.5 |
| Stearic (C18) | ≤11.0 | ≤6.0 | ≤0.5 |
| Oleic (C18:1) | ≤11.0 | ≥58.0 | ≥98.0 |
| Linoleic (C18:2) | n/a | ≤18.0 | ≤0.5 |
| Linolenic (C18:3) | ≤3.0 | ≤4.0 | ≤0.5 |
| Grades tested | HP PS20, | HP PS20, | POA |
| | SR PS20 | SR PS80 | PS80 |

In accordance with the present invention, the role of multivalent cations as nucleation factor for visible particle formation was investigated, and demonstrated in spiking studies with free fatty acid solutions as well as partially degraded polysorbates which were enzymatically hydrolyzed with different enzymes. Metal impurities, i.e. aluminum, calcium, magnesium, iron, zinc, can be introduced to a biopharmaceutical formulation through the manufacturing process (process leachables) (Zhou et al. 2011) or the primary packaging containers (glass leachables). (Ditter et al. 2018) Some of these metal impurities, e.g. iron, are known to promote oxidative degradation of polysorbates. (Kranz et al. 2019; Doyle Drbohlav et al. 2019)

Therefore, in one embodiment the present invention provides a stable aqueous composition comprising a protein together with pharmaceutically acceptable excipients such as, for example, buffers, stabilizers including antioxidants, and further comprising at least one chelator. In one embodiment, said stable aqueous composition (or preparation) is for parenteral use.

In another embodiment, the present invention provides a stable aqueous composition comprising a protein together with pharmaceutically acceptable excipients such as, for example, buffers, stabilizers including antioxidants, and further comprising free fatty acids, inorganic metal ions and at least one chelator. In one embodiment, said stable aqueous composition (or preparation) is for parenteral use. In another embodiment the free fatty acids are as defined herein. In yet another embodiment, said free fatty acids result from the hydrolytic degradation of PS20 or PS80. In yet another embodiment, said free fatty acids are present in said stable aqueous composition in concentrations below their solubility concentration and the concentration of the chelator is at least the same (i.e. equimolar) than the concentration of the inorganic metal ions. In this embodiment, the inorganic metal ions can be one or several ions selected from multivalent ions of aluminum, calcium, magnesium, iron and/or zinc, preferably aluminium or iron.

In one embodiment said “chelator” is selected from the group of Ethylenediaminetetraacetic acid (EDTA), Diethylenetriaminepentaacetic acid (DTPA or Pentetic Acid), Ethyleneglycol-bis(β-aminoethyl)-N,N,N′,N′-tetraacetic Acid (EGTA), N-Carboxymethyl-N′-(2-hydroxyethyl)-N,N′-ethylenediglycine (HEDTA), ethylenediamine-N,N′-bis(2-dihydroxyphenylacetic acid) (EDDHA), 1,3-Diaminopropane-N,N,N′,N′-tetraacetic acid (PDTA), Tetrasodium N,N-Bis(carboxymethyl)-L-glutamate (GLDA), citrate, malonate, tartrate, ascorbate, salicylic acid, aspartic acid, glutamic acid. In another embodiment, said chelator is Ethylenediaminetetraacetic acid (EDTA). In another embodiment said chelator is Diethylenetriaminepentaacetic acid (DTPA or Pentetic Acid). In still another embodiment only one chelator is used.

In one embodiment said chelator is present in a concentration from 0.0005 to 2.0% (w/v), or from 0.001 to 0.1% (w/v). In another embodiment, if the chelator is EDTA, it is present in a relative amount of 0.005% (w/v). In another embodiment, if the chelator is DTPA, it is present in an amount of 0.05 mM. In yet another embodiment, the chelator is present in at least the same (i.e. equimolar) amount than the metal impurities or inorganic metal ions in a composition according to the present invention.

In another embodiment, there is provided the composition as defined above, wherein the pH of said composition is in the range of 5 to 7. In one aspect the pH is about 5.5 or about 6.

In another embodiment, the present invention provides a composition as defined herein before, wherein the protein is an antibody. In one aspect, the antibody is a monoclonal antibody. In another aspect the antibody is a human or humanized monoclonal, mono- or bispecific antibody.

In yet another embodiment, the antibody in accordance with the present invention is the antibody with the INN pertuzumab. Pertuzumab is commercially available, for example under the tradename PERJETA®. Pertuzumab is, for example, also disclosed in EP 2 238 172 B1. Therefore, in another embodiment, “pertuzumab” (or “rhuMAb 2C4”) refer to an antibody comprising the variable light and variable heavy amino acid sequences in SEQ ID Nos. 3 and 4, respectfully as disclosed in EP 2 238 172 B1.

Where Pertuzumab is an intact antibody, it comprises the light chain and heavy chain amino acid sequences in SEQ ID Nos. 15 and 16, respectively as disclosed in EP 2 238 172 B1.

In another embodiment, the present invention provides a composition as defined herein before, consisting of the following components: Formulation A: 10 mg/mL API in 10 mM His/HisHCl, pH 5.0, 10 mM Methionine, 240 mM sucrose, 0.05% (w/v) PS20; Formulation B: 25 mg/mL API in 20 mM His, pH 6, 240 mM Trehalose, 0.02% (w/v) PS20, Formulation C: 50 mg/mL API in 20 mM L-His/His acetate buffer pH 5.5, 220 mM Sucrose, 10 mM L-Methionine, 0.04% (w/v) PS20, Formulation D: 180 mg/mL API in 20 mM L-His/His acetate buffer pH 5.5, 130 mM Arginine hydrochloride, 10 mM L-Methionine, 0.04% (w/v) PS20, Formulation E: 175 mg/mL API in 20 mM His/Asp pH 6.0, 150 mM Arginine, 40 mM Met, 0.05% (w/v) PS80. The term “API” as used herein means active pharmaceutical ingredient and is well known to the person of skill in the art of pharmaceutical preparations. In one embodiment, an API is a protein or antibody as defined herein.

In another embodiment, the present invention provides any of the compositions designated Formulation 01, 02, 03, 04 or 05 as specified in Example 5 (Table 7).

In another embodiment, the present invention provides a composition comprising pertuzumab at 30 mg/mL in 20 mM histidine acetate buffer (pH 6.0), 120 mM sucrose, 0.2 mg/mL HP PS20, 10 mM methionine and 0.05 mM DTPA.

In another embodiment, the present invention provides a composition comprising pertuzumab at 30 mg/mL in 20 mM histidine acetate buffer pH 6.0, 120 mM sucrose, 0.2 mg/mL HP PS20, 10 mM methionine and 0.05 mM EDTA.

In another embodiment, the present invention provides a composition comprising pertuzumab at 30 mg/mL in 20 mM histidine acetate buffer pH 6.0, 120 mM sucrose, 0.2 mg/mL pure oleic acid (POA) PS80, 10 mM methionine and 0.05 mM DTPA.

In another embodiment, the present invention provides a composition comprising pertuzumab at 30 mg/mL in 20 mM histidine acetate buffer pH 6.0, 120 mM sucrose, 0.2 mg/mL pure oleic acid (POA) PS80, 10 mM methionine and 0.05 mM EDTA. In another embodiment, the present invention provides the use of chelators, as defined herein, for the manufacture of medicaments, especially for the manufacture of stable parenteral protein-, more specifically parenteral antibody preparations. In one embodiment the parenteral preparation is an aqueous preparation. In another embodiment the parenteral preparation is for subcutaneous (sc) application. In another embodiment, the parenteral preparation is for intravenous (iv) application.

In another embodiment, the present invention provides the use of chelators, as defined herein, to prevent the formation of visible particles in parenteral protein, especially antibody preparations. In one aspect the parenteral preparation is an aqueous preparation. In another aspect the parenteral preparation is for subcutaneous (sc) application. In another aspect, the parenteral preparation is for intravenous (iv) application. In another aspect, the present invention provides the use of chelators, as defined herein, to prevent the formation of visible particles comprising free fatty acids in concentrations below their solubility level, in parenteral protein preparations.

The term “parenteral”, as used herein has its ordinary meaning. In one aspect parenteral means for subcutaneous (sc) injection and/or for intravenous injection.

The present parenteral protein preparations are “stable”, due to the presence of chelators, as defined herein. The term “stable” means that said preparations remains free; or essentially free; or practically free of visible particles until the end of their authorized shelf life. In one aspect the present preparations are stable for up to 30 months; or for up to 24 months; or for up to 18 months; or for up to 12 months. The stability of parenteral protein preparations can be affected by parameters well known to the skilled person such as, for example, light (UV radiation), temperature and/or shaking. Therefore, in one aspect, the term “stable” includes conditions usually recommended for storage of a product comprising the present parenteral protein-, or antibody preparation as, for example, described in the Summary of Product Characteristics (SmPC) issued by the European Medicins Agency (EMA) or the package insert for that given product. In one embodiment, the term “stable” includes a period of 30 months at a temperature between 2° C.-8° C. and substantially protected from light.

The presence of visible particles can be generally detected using methods as described in the European—or US Pharmacopoeia (see Ph.Eur 10.0; chapter 2.9.20; and First Supplement to USP 37-NF 32 <790>). In one embodiment of the present invention, the term “free” of visible particles means that no visible particle can be detected in a parenteral protein preparation using the method described in the accompanying working examples, utilizing a Seidenader V 90-T instrument (Seidenader Maschinenbau GmbH. Markt Schwaben. DE). The term “essentially free” of visible particles means that 1 to 5 visible particles can be detected in a parenteral protein preparation using the method and conditions described in the accompanying working examples, utilizing a Seidenader V 90-T instrument (Seidenader Maschinenbau GmbH, Markt Schwaben, DE). The term “practically free” of visible particles means that 0 to 4 visible particles can be detected using a black and white panel (herein “E/P box” or “E/P”) as described in the European Pharmacopoeia (see Ph.Eur 10.0; chapter 2.9.20).

The term “visible particles” means particles comprising one or several free fatty acids, or mixtures of aggregated protein and free fatty acids. In one aspect visible particles have a particle size of at least 80 μm, or at least 100 μm and can, for example, be seen as turbidity or precipitate in a parenteral protein preparation. In one embodiment, the visible particles form with at least one multivalent cation and free fatty acids cleaved from surfactants present in the parenteral protein preparation such as, e.g. PS20 and PS80. The term “multivalent cation(s)”, as used herein, means one or several metal impurities which is/are introduced to a parenteral protein preparation through the manufacturing process or the primary packaging containers. In one embodiment such multivalent cation is a cation selected from aluminum, calcium, magnesium, iron, zinc. The term “fatty acid”, as used herein has its ordinary meaning known to a person of skill in Organic Chemistry. In one embodiment, the term fatty acid means any fatty acid(s) present in—or cleaved from PS20 or PS80. In another embodiment the term “fatty acid” means lauric acid, or myristic acid, or palmitic acid, or stearic acid, or oleic acid. In accordance with the present invention, said fatty acids can be present in the aqueous protein preparations at concentrations below their solubility concentration (or “solubility level”) and form visible particles together with the multivalent cations as defined herein, as nucleation factors. The solubility concentrations of the fatty acids as defined herein are well known to the skilled person and can for example be found in (Doshi et al. 2015; Doshi et al. 2020b; Glucklich et al. 2020). In one embodiment the term “below their solubility concentration” means below the solubility concentration of the fatty acids as defined herein in aqueous solution, or buffer, at any temperature between 0° C. and 30° C. In another embodiment the term “below their solubility concentration” means below the solubility concentration of the fatty acids as defined herein in aqueous solution, or buffer, at a temperature of 2-8° C. In yet another embodiment the term “below their solubility concentration” means below the solubility concentration of the fatty acids as defined herein in aqueous solution, or buffer, at a temperature of about 5° C.

Therefore, in yet another embodiment, the present invention provides the use of chelators, as defined herein, for the manufacture of medicaments, especially for the manufacture of aqueous parenteral protein-, more specifically parenteral antibody preparations, which are characterized in that they remain free, or practicable free, or essentially free of visible particles comprising free fatty acids resulting from degradation of PS20 or PS80 and, optionally, one or several multivalent cation(s), for the entire time of their authorized shelf life, but at least for up to 30 months; or for up to 24 months; or for up to 18 months; or for up to 12 months and under conditions recommended for storage of such preparations.

In another embodiment, the present invention provides a pharmaceutical dosage form comprising a protein preparation as defined herein, for example an aqueous antibody preparation in a container such as, for example, a vial or syringe.

In another embodiment, the present invention provides a pharmaceutical dosage form comprising a protein preparation obtained from the use of chelators as defined herein in a container such as, for example, a vial or syringe.

The term “excipient” refers to an ingredient in a pharmaceutical composition or preparation, other than an active ingredient, which is nontoxic to a subject. An excipient includes, but is not limited to, a buffer, stabilizer including antioxidant or preservative.

The term “buffer” is well known to a person of skill in the art of organic chemistry or pharmaceutical sciences such as, for example, pharmaceutical preparation development. Buffer as used herein means acetate, succinate, citrate, arginine, histidine, phosphate, Tris, glycine, aspartate, and glutamate buffer systems.

Furthermore, within this embodiment, the histidine concentration of said buffer is from 5 to 50 mM. Preferred buffers are free histidine base and histidine-HCl or acetate or succinate and/or aspartate. Furthermore, within this embodiment, the histidine concentration of said buffer is from 5 to 50 mM.

The term “stabilizer” is well known to a person of skill in the art of organic chemistry or pharmaceutical sciences such as, for example, pharmaceutical preparation development. A stabilizer in accordance with the present invention is selected from the group consisting of sugars, sugar alcohols, sugar derivatives, or amino acids. In one aspect the stabilizer is (1) sucrose, trehalose, cyclodextrines, sorbitol, mannitol, glycine, or/and (2) methionine, and/or (3) arginine, or lysine. In still another aspect, the concentration of said stabilizer is (1) up to 500 mM or (2) 5-25 mM, or/and (3) up to 350 mM, respectively The term “protein” as used herein means any therapeutically relevant polypeptide. In one embodiment, the term protein means an antibody. In another embodiment, the term protein means an immunocunjugate.

The term “antibody” herein is used in the broadest sense and encompasses various antibody classes or structures, including but not limited to monoclonal antibodies, polyclonal antibodies, multispecific antibodies (e.g., bispecific antibodies), and antibody fragments so long as they exhibit the desired antigen-binding activity. In one embodiment, any of these antibodies is human or humanized. In one aspect, the antibody is selected from alemtuzumab (LEMTRADA®), atezolizumab (TECENTRIQ®), bevacizumab (AVASTIN®), cetuximab (ERBITUX®), panitumumab (VECTIBIX@), pertuzumab (OMNITARG/PERJETA®, 2C4), trastuzumab (HERCEPTIN@), tositumomab (Bexxar®), abciximab (REOPRO®), adalimumab (HUMIRA®), apolizumab, aselizumab, atlizumab, bapineuzumab, basiliximab (SIMULECT®), bavituximab, belimumab (BENLYSTA®) briankinumab, canakinumab (ILARIS®), cedelizumab, certolizumab pegol (CIMZIA®), cidfusituzumab, cidtuzumab, cixutumumab, clazakizumab, crenezumab, dacizumab (ZENAPAX®), dalotuzumab, denosumab (PROLIA®, XGEVA®), eculizumab (SOLIRIS®), efalizumab, epratuzumab, erlizumab, emicizumab (HEMLIBRA®), felvizumab, fontolizumab, golimumab (SIMPONI®), ipilimumab, imgatuzumab, infliximab (REMICADE®), labetuzumab, lebrikizumab, lexatumumab, lintuzumab, lucatumumab, lulizumab pegol, lumretuzumab, mapatumumab, matuzumab, mepolizumab, mogamulizumab, motavizumab, motovizumab, muronomab, natalizumab (TYSABRI®), necitumumab (PORTRAZZA®), nimotuzumab (THERACIM®), nolovizumab, numavizumab, olokizumab, omalizumab (XOLAIR®), onartuzumab (also known as MetMAb), palivizumab (SYNAGIS®), pascolizumab, pecfusituzumab, pectuzumab, pembrolizumab (KEYTRUDA®), pexelizumab, priliximab, ralivizumab, ranibizumab (LUCENTIS®), reslivizumab, reslizumab, resyvizumab, robatumumab, rontalizumab, rovelizumab, ruplizumab, sarilumab, secukinumab, seribantumab, sifalimumab, sibrotuzumab, siltuximab (SYLVANT®) siplizumab, sontuzumab, tadocizumab, talizumab, tefibazumab, tocilizumab (ACTEMRA®), toralizumab, tucusituzumab, umavizumab, urtoxazumab, ustekinumab (STELARA®), vedolizumab (ENTYVIO®), visilizumab, zanolimumab, zalutumumab.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the IgG1 isotype. In certain aspects, the antibody is of the IgG1 isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG4 antibody. The heavy chain constant domains that correspond to the different classes of immunoglobulins are called a, d, e, g, and m, respectively. The light chain of an antibody may be assigned to one of two types, called kappa (x) and lambda (k), based on the amino acid sequence of its constant domain.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “pharmaceutical composition” or “pharmaceutical preparation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or preparation, other than an active ingredient, which is nontoxic to a subject. A pharmaceutically acceptable carrier includes, but is not limited to an excipient as defined herein.

A. Chimeric and Humanized Antibodies

In certain aspects, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer. 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)): framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

B. Human Antibodies

In certain aspects, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat.

Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

C. Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

D. Immunoconjugates

The invention also provides immunoconjugates comprising an antibody herein conjugated (chemically bound) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At211, I131, I125, Y90, Re186, Re188, Sm153, Bi212, P32, Pb212 and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP. SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford. Ill., U.S.A).

E. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172. WO 2010/145792, and WO 2013/026831. The bispecific antibody or antigen binding fragment thereof also includes a “Dual Acting FAb” or “DAF” comprising an antigen binding site that binds to two different antigens, or two different epitopes of the same antigen (see. e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

F. Recombinant Methods and Compositions

Antibodies may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised in one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S. L368A and Y407V and optionally Y349C or S354C) (see, e.g., Carter, P. et al., Immunotechnol. 2 (1996) 73) according to EU index numbering.

For recombinant production of an antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248. Lo, B. K. C. (ed.), Humana Press. Totowa, N.J. (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CVI); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described. e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J. (2004), pp. 255-268.

The invention will now be further illustrated by the following, non-limiting working examples.

### EXAMPLES

### Materials and Methods

Solubility of Free Fatty Acids in Aqueous Buffer Solution

Free fatty acid stock solutions were prepared as previously described by Doshi et al (Doshi et al. 2015) with slight modifications. In brief, lauric acid (“LA”. Sigma-Aldrich/Merck, Darmstadt, DE) and myristic acid (“MA”, Sigma-Aldrich/Merck, Darmstadt, DE) were suspended in PS20 HP (Croda, Edison, N.J., USA) and stirred (150 rpm) for 30 min at 60° C. until both FFAs were fully dissolved. Solutions were diluted 1:5 with pre-warmed (60° C.) water for injection (WFI) and immediately filtered through 0.22 μm PVDF Steriflip filters (Merck Millipore, Darmstadt, DE). The concentration of LA and MA in FFA stock solutions was verified by LC-MS as described by Honemann et al. (Honemann et al. 2019) LA/MA/PS20 stock solutions were spiked into 20 mM histidine acetate buffer pH 5.5 (1:500 dilution, n=3) and homogenized on a MaxQ™ 4000 Benchtop Orbital Shaker (Thermo Scientific™, Waltham, Mass., USA) for 1 hour at 25° C. Samples were stored at 5° C. and analyzed for visible particles after 0, 1, 7 and 28 days using a black/white panel according Ph. Eur. 2.9.20 (European Directorate for the Quality of Medicines) and a Seidenader V 90-T instrument (Seidenader Maschinenbau GmbH, Markt Schwaben, DE). All samples were equilibrated to ambient temperature (1 hour) prior visual inspection. The number of visible particles per container was defined as ‘many particles (>7)’, ‘few particles (5-7)’, or ‘practically free of particles (0-4)’ in E/P box, and ‘many particles (>10)’, ‘few particles (6-10)’, ‘essentially free of particles (1-5)’, or ‘free of particles (0)’ by Seidenader.

The composition of FFA stock solutions and samples is outlined in Table 1.

TABLE 1
Composition of LA/MA/PS20 stock solutions and samples

| | Concentration (stock | | | Target concentration in | | |
|---|---|---|---|---|---|---|
| | solution) mg/mL | | | sample μg/mL | | |
| FFA stock | lauric | myristic | | lauric | myristic | |
| solution | acid | acid | matrix | acid | acid | matrix |
| LAMA-1 | 15 | 6 | 20% | 30 | 12 | 0.04% |
| LAMA-2 | 12.5 | 5 | (w/v) | 25 | 10 | (w/v) PS20 |
| LAMA-3 | 10 | 4 | PS20 in | 20 | 8 | in 20 mM |
| LAMA-4 | 7.5 | 3 | WFI | 15 | 6 | histidine |
| LAMA-5 | 6.25 | 2.5 | | 12.5 | 5 | acetate |
| LAMA-6 | 5 | 2 | | 10 | 4 | buffer pH |
| LAMA-7 | 3.75 | 1.5 | | 7.5 | 3 | 5.5 |
| LAMA-8 | 2.5 | 1 | | 5 | 2 | |
| LAMA-9 | 1.25 | 0.5 | | 2.5 | 1 | |
| LAMA-10 | 0 | 0 | | 0 | 0 | |

Spiking Study with Free Fatty Acids and Aluminum

A 100 ppm aluminum stock solution was prepared from aluminum chloride hexahydrate in 20 mM histidine acetate pH 5.5. The actual concentration of aluminum was determined by inductively-coupled plasma mass spectrometry (ICP-MS). This stock solution was then diluted to 10 ppm Al³⁺ and sterile-filtered through a 0.22 μm porosity filter cartridge (Sterivex-GV, Millipore). Further dilutions (10-250 ppb Al³⁺) were prepared aseptically under laminar air flow and dispensed into 20 mL type I borosilicate glass vials (Schott. Mainz, DE).

Samples containing different amounts of aluminum (0-250 ppb) were spiked with different FFA stock solutions (LAMA-2, LAMA-6, LAMA-7, LAMA-8 and LAMA-10). Samples containing 250 ppb aluminum were additionally spiked with ethylenediaminetetraacetic acid (EDTA) to attain a target concentration of 0.005% (w/v). All vials were sealed with 20 mm teflonized injection stoppers (D777-1, Daikyo) and aluminum crimp caps and homogenized on a MaxQ™ 4000 Benchtop Orbital Shaker (Thermo Scientific™, Waltham, Mass., USA) for 1 hour at 25° C.

Dilutions spiked with LAMA-6, LAMA-7 and LAMA-8 resulted in LA and MA concentrations below their solubility limit, whereas spiking with LAMA-2 yielded FFA concentrations above the solubility limit and served as positive control. LAMA-10 only comprised polysorbate 20 and was used for the preparation of negative controls. All samples were prepared in triplicates.

Samples were stored at 5° C. and the formation of visible particles was assessed for up to 28 days using a black/white and a Seidenader V 90-T instrument (Seidenader Maschinenbau GmbH, Markt Schwaben, DE) as described above.

Solubility of Partially Degraded PS20

PS20 was enzymatically hydrolyzed with immobilized enzymes (Graf et al. 2020) (mucor miehei lipase (MML) and Candida antarctica lipase (CAL)) possessing different specificities towards mono- and higher-order esters. A set of polysorbates with six different degradation levels (10%, 15%, 20%, 30%, 40% and 60% degradation) was prepared for each enzyme (Table 2).

PS20 stock solutions (50 mg/mL) were spiked into 20 mM histidine buffer pH 5.5 (1:125 dilution, n=3) and homogenized on a MaxQ™ 4000 Benchtop Orbital Shaker (Thermo Scientific™, Waltham, Mass., USA) for 1 hour at 25° C. Samples were stored at 5° C. and analyzed for visible particles after 0, 1, 7 and 28 days using a black/white panel according Ph. Eur. 2.9.20 (European Directorate for the Quality of Medicines). All samples were equilibrated to ambient temperature (1 hour) prior visual inspection. The number of visible particles per container was defined as ‘many particles (>7)’, ‘few particles (5-7)’, or ‘practically free of particles (0-4)’.

TABLE 2
Enzymatically degraded polysorbates

| PS20 stock solution | Enzyme | Rel. Degradation | Intact PS20 |
|---|---|---|---|
| PS20-Std | n/a | 0% | 100% |
| MML-10 | MML | 10% | 90% |
| MML-15 | | 15% | 85% |
| MML-20 | | 20% | 80% |
| MML-30 | | 30% | 70% |
| MML-40 | | 40% | 60% |
| MML-60 | | 60% | 40% |
| CAL-10 | CAL | 10% | 90% |
| CAL-15 | | 15% | 85% |
| CAL-20 | | 20% | 80% |
| CAL-30 | | 30% | 70% |
| CAL-40 | | 40% | 60% |
| CAL-60 | | 60% | 40% |

Spiking studies with partially degraded polysorbate 20 and aluminum

Diluted aluminum solutions comprising in 20 mM histidine acetate pH 5.5 were prepared as described above and filled into 20 mL type I borosilicate glass vials (Schott, Mainz, DE). Samples containing different amounts of aluminum (0-250 ppb) were spiked with different PS20 stock solutions (PS20-Std, MML-10, MML-15, MML-40, CAL-10, CAL-15). In addition, samples containing 250 ppb aluminum were supplemented with 0.005% (w/v) ethylenediaminetetraacetic acid (EDTA) or 0.05 mM diethylenetriaminepentaacetic acid (DTPA) prior spiking with partially degraded PS20. Vials were sealed with 20 mm teflonized injection stoppers (D777-1, Daikyo) and aluminum crimp caps and homogenized on a MaxQ™ 4000 Benchtop Orbital Shaker (Thermo Scientific™, Waltham, Mass., USA) for 1 hour at 25° C.

Samples prepared with MML-10/-15 and CAL-10/-15 resulted in FFA concentrations below their solubility limit, whereas spiking with MML-40 (positive controls) resulted in FFA concentrations above the solubility limit. PS20-Std only comprised non-degraded polysorbate 20 and was used for the preparation of negative controls. All samples were prepared in triplicates.

Samples were stored at 5° C. and particle formation was assessed for up to 28 days by visual inspection using a black/white panel according Ph. Eur. 2.9.20. (European Directorate for the Quality of Medicines)

### Example 1: Solubility of Free Fatty Acids in Histidine Acetate Buffer pH 5.5

Free fatty acid (FFA) stock solutions of lauric acid (LA) and myristic acid (MA) were spiked into 20 mM histidine acetate buffer pH 5.5 (n=3). Samples were analyzed for the presence of visible particles after incubation at 2-8° C. for 0, 1 day, 7 days and 28 days using the (A) Seidenader and (B) E/P box. The number of particles per container was classified as many particles ‘(>7, xxx)’, few particles ‘(5-7, xx)’, or practically free of particles ‘(0-4, /)’ in E/P box, and ‘many particles (>10, xxx)’, ‘few particles (6-10, xx)’, ‘essentially free of particles (1-5, x)’, or ‘free of particles (0, /)’ by Seidenader. d=day of inspection, d0=after spiking. The results are summarized in Table 3.

TABLE 3
Solubility of free fatty acids in histidine acetate buffer pH 5.5.

| | FFA | | | | | | | | | | | | | |
|---|---|---|---|---|---|---|---|---|---|---|---|---|---|---|
| | target concentrations | | | | | | | | | | | | | |
| | Lauric | Myristic | d0 | | | d1 | | | d7 | | | d28 | | |
| | acid | acid | Vial | | | Vial | | | Vial | | | Vial | | |
| Sample | (μg/mL) | (μg/mL) | #1 | #2 | #3 | #1 | #2 | #3 | #1 | #2 | #3 | #1 | #2 | #3 |

A

| | | | | | | | | | | | | | | |
|---|---|---|---|---|---|---|---|---|---|---|---|---|---|---|
| 1 | 30 | 12 | / | / | / | xxx | xxx | xxx | xxx | xxx | xxx | xxx | xxx | xxx |
| 2 | 25 | 10 | / | / | / | xxx | xxx | xxx | xxx | xxx | xxx | xxx | xxx | xxx |
| 3 | 20 | 8 | / | / | x | xxx | xxx | xxx | xxx | xxx | xxx | xxx | xxx | xxx |
| 4 | 15 | 6 | / | x | x | x | x | x | xxx | xxx | xxx | xxx | xxx | xxx |
| 5 | 12.5 | 5 | / | / | / | / | x | x | xx | x | xx | xxx | xxx | xxx |
| 6 | 10 | 4 | / | / | / | / | / | / | x | x | xx | xxx | xx | xx |
| 7 | 7.5 | 3 | / | / | / | / | / | / | x | x | x | xx | xx | x |
| 8 | 5 | 2 | / | / | x | / | / | / | x | x | x | x | x | x |
| 9 | 2.5 | 1 | x | / | / | / | / | / | / | / | / | x | x | x |
| 10 | 0 | 0 | / | / | x | / | / | / | / | / | / | / | / | / |

B

| | | | | | | | | | | | | | | |
|---|---|---|---|---|---|---|---|---|---|---|---|---|---|---|
| 1 | 30 | 12 | / | / | / | xxx | xxx | xxx | xxx | xxx | xxx | xxx | xxx | xxx |
| 2 | 25 | 10 | / | / | / | xxx | xxx | xxx | xxx | xxx | xxx | xxx | xxx | xxx |
| 3 | 20 | 8 | / | / | / | xxx | xxx | xxx | xxx | xxx | xxx | xxx | xxx | xxx |
| 4 | 15 | 6 | / | / | / | / | / | / | xx | xxx | / | xxx | xxx | xxx |
| 5 | 12.5 | 5 | / | / | / | / | / | / | / | / | / | xx | / | / |
| 6 | 10 | 4 | / | / | / | / | / | / | / | / | / | / | / | / |
| 7 | 7.5 | 3 | / | / | / | / | / | / | / | / | / | / | / | / |
| 8 | 5 | 2 | / | / | / | / | / | / | / | / | / | / | / | / |
| 9 | 2.5 | 1 | / | / | / | / | / | / | / | / | / | / | / | / |
| 10 | 0 | 0 | / | / | / | / | / | / | / | / | / | / | / | / |

### Example 2: Visible Particle Formation

After spiking of FFA stock solutions into aqueous buffered solutions (20 mM histidine acetate buffer pH 5.5) containing different amounts of aluminum ranging from 0 to 250 ppb (n=3). Samples containing the highest amount of aluminum (250 ppb) were additionally formulated with a chelator (EDTA). Samples were analyzed for the presence of visible particles after storage at 2-8° C. for 0, 1 day, 7 days and 28 days using the (A) Seidenader and (B) E/P box. The cumulative content of particles per container was classified as ‘many particles (>7, xxx)’, ‘few particles (5-7, xx)’, or ‘practically free of particles (0-4,/)’ in E/P box, and ‘many particles (>10, xxx)’, ‘few particles (6-10, xx)’, ‘essentially free of particles (1-5, x)’, or ‘free of particles (0, /)’ by Seidenader. Sample containing either no FFA or no salt were used as negative controls, whereas a sample containing FFAs above the solubility limit (*) was used as positive controls. d=day of inspection, d0=after spiking. nd=not determined. The results are summarized in Table 4.

TABLE 4
Visible particle formation

| Sample composition | | | | d0 | | | d7 | | | d14 | | | d28 | | |
|---|---|---|---|---|---|---|---|---|---|---|---|---|---|---|---|
| LA | MA | Al³⁺ | EDTA | Vial | | | Vial | | | Vial | | | Vial | | |
| μg/mL | μg/mL | ppb | % (w/v) | #1 | #2 | #3 | #1 | #2 | #3 | #1 | #2 | #3 | #1 | #2 | #3 |

A

| | | | | | | | | | | | | | | | | |
|---|---|---|---|---|---|---|---|---|---|---|---|---|---|---|---|---|
| 1 | 10 | 4 | 250 | 0.005 | / | / | / | / | / | / | / | / | / | / | / | / |
| 2 | | | 250 | — | / | / | / | / | / | / | xxx | xxx | xxx | xxx | xxx | xxx |
| 3 | | | 125 | — | / | / | / | / | / | / | / | / | / | / | / | / |
| 4 | | | 100 | — | / | / | / | / | / | / | / | / | / | / | / | / |
| 5 | | | 75 | — | / | / | / | / | / | / | / | / | / | / | / | / |
| 6 | | | 50 | — | / | / | / | / | / | / | / | / | / | / | / | / |
| 7 | | | 25 | — | / | / | / | / | / | / | / | / | / | / | / | / |
| 8 | | | 10 | — | nd | nd | nd | / | / | / | / | / | / | / | / | / |
| 9 | | | 0 | — | / | / | / | / | / | / | / | / | / | / | / | / |
| 10 | 7.5 | 3 | 250 | 0.005 | / | / | / | / | / | / | / | / | / | / | / | / |
| 11 | | | 250 | — | xx | / | / | xxx | xxx | xxx | xxx | xxx | xxx | xxx | xxx | xxx |
| 12 | | | 125 | — | / | / | / | xxx | xxx | xxx | / | / | / | / | / | / |
| 13 | | | 100 | — | / | / | / | / | / | / | / | / | / | / | / | / |
| 14 | | | 75 | — | / | / | / | / | / | / | / | / | / | / | / | / |
| 15 | | | 50 | — | / | / | / | / | / | / | / | / | / | / | / | / |
| 16 | | | 25 | — | / | / | / | / | / | / | / | / | / | / | / | / |
| 17 | | | 10 | — | nd | nd | nd | / | / | / | / | / | / | / | / | / |
| 18 | | | 0 | — | / | / | / | / | / | / | / | / | / | / | / | / |
| 19 | 5 | 2 | 250 | 0.005 | / | / | / | / | / | / | / | / | / | / | / | / |
| 20 | | | 250 | — | / | / | / | xxx | xxx | xxx | xxx | xxx | xxx | xxx | xxx | xxx |
| 21 | | | 125 | — | / | / | / | / | / | / | xxx | xxx | xxx | / | / | / |
| 22 | | | 100 | — | / | / | / | xxx | xxx | xxx | xxx | xxx | xxx | xxx | xxx | xxx |
| 23 | | | 75 | — | / | / | / | / | / | / | / | / | / | xxx | xxx | xxx |
| 24 | | | 50 | — | / | / | / | / | / | / | / | / | / | / | / | / |
| 25 | | | 25 | — | / | / | / | / | / | / | / | / | / | / | / | / |
| 26 | | | 10 | — | nd | nd | nd | / | / | / | / | / | / | / | / | / |
| 27 | | | 0 | — | / | / | / | / | / | / | / | / | / | / | / | / |
| 28 | 0 | 0 | 250 | — | nd | nd | nd | / | / | / | / | / | / | / | / | / |
| 29 | | | 125 | — | nd | nd | nd | / | / | / | / | / | / | / | / | / |
| 30 | | | 100 | — | nd | nd | nd | / | / | / | / | / | / | / | / | / |
| 31 | | | 75 | — | nd | nd | nd | / | / | / | / | / | / | / | / | / |
| 32 | | | 50 | — | nd | nd | nd | / | / | / | / | / | / | / | / | / |
| 33 | | | 25 | — | nd | nd | nd | / | / | / | / | / | / | / | / | / |
| 34 | | | 10 | — | nd | nd | nd | / | / | / | / | / | / | / | / | / |
| 35 | | | 0 | — | / | / | / | / | / | / | / | / | / | / | / | / |
| 36 | 25* | 10* | 0 | — | / | / | / | xxx | xxx | xxx | xxx | xxx | xxx | xxx | xxx | xxx |

B

| | | | | | | | | | | | | | | | | |
|---|---|---|---|---|---|---|---|---|---|---|---|---|---|---|---|---|
| 1 | 10 | 4 | 250 | 0.005 | x | x | x | x | x | x | x | x | x | x | xx | x |
| 2 | | | 250 | — | xxx | xxx | xxx | xxx | xxx | xxx | xxx | xxx | xxx | xxx | xxx | xxx |
| 3 | | | 125 | — | xxx | xxx | xxx | xxx | xxx | xxx | xxx | xxx | xxx | xxx | xxx | xxx |
| 4 | | | 100 | — | xxx | xxx | xxx | xxx | xxx | xxx | xxx | xxx | xxx | xxx | xxx | xxx |
| 5 | | | 75 | — | xxx | xxx | xxx | xxx | xxx | xxx | xxx | xxx | xxx | xxx | xxx | xxx |
| 6 | | | 50 | — | x | xxx | xx | xxx | xxx | xxx | xxx | xxx | xxx | xxx | xxx | xxx |
| 7 | | | 25 | — | / | x | / | xxx | xxx | xxx | xxx | xxx | xxx | xxx | xxx | xxx |
| 8 | | | 10 | — | x | x | x | xxx | xxx | xxx | xx | xxx | xxx | xxx | xxx | xxx |
| 9 | | | 0 | — | / | x | x | x | x | x | x | / | x | x | x | / |
| 10 | 7.5 | 3 | 250 | 0.005 | / | / | / | x | x | x | x | x | x | / | x | / |
| 11 | | | 250 | — | xxx | xxx | xxx | xxx | xxx | xxx | xxx | xxx | xxx | xxx | xxx | xxx |
| 12 | | | 125 | — | xxx | xxx | xxx | xxx | xxx | xxx | xxx | xxx | xxx | xxx | xxx | xxx |
| 13 | | | 100 | — | xxx | xxx | xxx | xxx | xxx | xxx | xxx | xxx | xxx | xxx | xxx | xxx |
| 14 | | | 75 | — | xxx | xxx | xxx | xxx | xxx | xxx | xxx | xxx | xxx | xxx | xxx | xxx |
| 15 | | | 50 | — | xxx | xx | xxx | xxx | xxx | xxx | xxx | xxx | xxx | xxx | xxx | xxx |
| 16 | | | 25 | — | x | x | / | xxx | xxx | xxx | xxx | xxx | xxx | xxx | xxx | xxx |
| 17 | | | 10 | — | x | / | / | xxx | xxx | xxx | x | xxx | xx | x | xxx | xx |
| 18 | | | 0 | — | / | / | / | x | x | xxx | x | / | xxx | x | x | xxx |
| 19 | 5 | 2 | 250 | 0.005 | x | / | x | x | xx | xx | x | x | x | x | x | / |
| 20 | | | 250 | — | xxx | xxx | xxx | xxx | xxx | xxx | xxx | xxx | xxx | xxx | xxx | xxx |
| 21 | | | 125 | — | xxx | xxx | xxx | xxx | xxx | xxx | xxx | xxx | xxx | xxx | xxx | xxx |
| 22 | | | 100 | — | xxx | xxx | xxx | xxx | xxx | xxx | xxx | xxx | xxx | xxx | xxx | xxx |
| 23 | | | 75 | — | x | x | x | xxx | xxx | xxx | xxx | xxx | xxx | xxx | xxx | xxx |
| 24 | | | 50 | — | / | / | / | xx | / | / | / | / | / | / | / | / |
| 25 | | | 25 | — | / | / | / | xxx | xxx | xxx | xxx | xxx | xxx | xxx | xxx | xxx |
| 26 | | | 10 | — | / | x | / | xxx | xxx | xxx | xxx | xxx | xx | xxx | xxx | xx |
| 27 | | | 0 | — | / | / | / | x | x | / | / | / | x | / | / | / |
| 28 | 0 | 0 | 250 | — | / | x | / | xxx | xxx | xxx | x | xx | xx | xx | x | xx |
| 29 | | | 125 | — | / | x | / | / | / | x | / | / | / | / | x | x |
| 30 | | | 100 | — | / | x | / | x | x | / | x | x | / | x | / | x |
| 31 | | | 75 | — | / | x | x | / | x | / | x | / | / | / | / | / |
| 32 | | | 50 | — | x | / | x | / | x | x | x | x | x | / | x | / |
| 33 | | | 25 | — | / | x | / | x | x | x | x | x | x | x | x | x |
| 34 | | | 10 | — | / | / | / | x | x | x | x | xx | x | x | x | x |
| 35 | | | 0 | — | / | x | x | x | x | x | x | xx | x | x | x | x |
| 36 | 25* | 10* | 0 | — | xxx | xxx | xxx | xxx | xxx | xxx | xxx | xxx | xxx | xxx | xxx | xxx |

### Example 3: Solubility of Partially Degraded Polysorbate 20 in Histidine Acetate Buffer pH 5.5

Polysorbate 20 (PS20) which was previously degraded by MML or CAL (degradation level 0-60%) was spiked into 20 mM histidine acetate buffer pH 5.5 (n=3) to a final PS20 concentration of 0.04% (w/v). Samples were visually inspected (E/P box) for the presence of visible particles after storage at 2-8° C. for 0, 1 day, 7 days and 28 days. The cumulative content of particles per container was classified as ‘many particles (>7, xxx)’, ‘few particles (5-7, xx)’, or ‘practically free of particles (0-4, /)’ in F/P box. d=day of inspection, d0=after spiking, MML=mucor miehei lipase, CAL=Candida antarctica lipase. The results are summarized in Table 5.

TABLE 5
Solubility of partially degraded polysorbate 20 in histidine acetate buffer pH 5.5.

| | partially degraded | | d0 | | | d1 | | | d7 | | | d28 | | |
|---|---|---|---|---|---|---|---|---|---|---|---|---|---|---|
| | polysorbate 20 | | Vial | | | Vial | | | Vial | | | Vial | | |
| Sample | enzyme | degradation | #1 | #2 | #3 | #1 | #2 | #3 | #1 | #2 | #3 | #1 | #2 | #3 |
| 1 | n/a | 0% | / | / | / | / | / | / | / | / | / | / | / | / |
| 2 | MML | 10% | / | / | / | / | / | / | / | / | / | / | / | / |
| 3 | | 15% | / | / | / | / | / | / | / | / | / | / | / | / |
| 4 | | 20% | / | / | / | xxx | xxx | xxx | xxx | xxx | xxx | xxx | xxx | xxx |
| 5 | | 30% | / | / | / | xxx | xxx | xxx | xxx | xxx | xxx | xxx | xxx | xxx |
| 6 | | 40% | xxx | xxx | xxx | xxx | xxx | xxx | xxx | xxx | xxx | xxx | xxx | xxx |
| 7 | | 60% | xxx | xxx | xxx | xxx | xxx | xxx | xxx | xxx | xxx | xxx | xxx | xxx |
| 8 | CAL | 10% | / | / | / | / | / | / | / | / | / | / | / | / |
| 9 | | 15% | / | / | / | / | / | / | / | / | / | / | / | / |
| 10 | | 20% | / | / | / | / | / | / | / | / | / | / | / | / |
| 11 | | 30% | / | / | / | / | / | / | xxx | xxx | xxx | xxx | xxx | xxx |
| 12 | | 40% | / | / | / | xx | xxx | xx | xxx | xxx | xxx | xxx | xxx | xxx |
| 13 | | 60% | / | / | / | xxx | xxx | xxx | xxx | xxx | xxx | xxx | xxx | xxx |

### Example 4: Visible Particle Formation

After spiking of partially degraded polysorbate 20 into aqueous buffered solutions (20 mM histidine acetate buffer pH 5.5) containing different amounts of aluminum ranging from 0 to 250 ppb (n=3). Samples containing the highest amount of aluminum (250 ppb) were additionally formulated with a chelator (EDTA or DTPA). Samples were visually inspected (E/P box) for the presence of visible particles after incubation for 0, 1 day, 7 days and 28 days at 2-8° C. The cumulative content of particles per container was classified as ‘many particles (>7, xxx)’, ‘few particles (5-7, xx)’, or ‘practically free of particles (0-4, /)’. Sample with 100% intact PS20 or without salt were used as negative controls, whereas samples containing 60% degraded PS (MML) above the solubility limit (*) was used as positive control. d=day of inspection. d0=after spiking, nd=not determined, MML=mucor miehei lipase, CAL=Candida antarctica lipase. The results are summarized in Table 6.

TABLE 6
Visible particle formation

| | Sample composition | | | | | | | | | | | | | | | |
|---|---|---|---|---|---|---|---|---|---|---|---|---|---|---|---|---|
| | | | EDTA | | d0 | | | d7 | | | d14 | | | d28 | | |
| | | Al³⁺ | % | DTPA | Vial | | | Vial | | | Vial | | | Vial | | |
| | PS20 | ppb | (w/v) | mM | #1 | #2 | #3 | #1 | #2 | #3 | #1 | #2 | #3 | #1 | #2 | #3 |
| 1 | MML- | 250 | 0.005 | — | nd | nd | nd | / | / | / | / | / | / | xx | / | / |
| 2 | 10 | 250 | — | 0.05 | nd | nd | nd | / | / | / | / | / | / | / | / | / |
| 3 | | 250 | — | — | nd | nd | nd | / | / | xxx | / | / | / | xxx | xxx | xxx |
| 4 | | 100 | — | — | nd | nd | nd | / | xxx | / | / | xxx | / | xxx | xxx | xxx |
| 5 | | 50 | — | — | nd | nd | nd | / | / | / | / | / | / | xxx | xxx | xxx |
| 6 | | 10 | — | — | nd | nd | nd | / | / | / | / | / | / | / | / | / |
| 7 | | 0 | — | — | nd | nd | nd | / | / | / | / | / | / | / | / | / |
| 8 | MML- | 250 | 0.005 | — | nd | nd | nd | / | / | / | / | / | / | xx | / | / |
| 9 | 15 | 250 | — | 0.05 | nd | nd | nd | / | / | / | / | / | / | / | / | / |
| 10 | | 250 | — | — | nd | nd | nd | / | / | / | / | / | / | xxx | xxx | xxx |
| 11 | | 100 | — | — | nd | nd | nd | / | / | / | / | / | / | / | / | / |
| 12 | | 50 | — | — | nd | nd | nd | / | / | / | / | / | / | / | / | / |
| 13 | | 10 | — | — | nd | nd | nd | / | / | / | / | / | xx | / | / | / |
| 14 | | 0 | — | — | nd | nd | nd | / | / | / | / | / | / | / | / | / |
| 15 | CAL- | 250 | 0.005 | — | nd | nd | nd | / | / | / | / | / | / | / | / | / |
| 16 | 10 | 250 | — | 0.05 | nd | nd | nd | / | / | / | / | / | / | / | / | / |
| 17 | | 250 | — | — | nd | nd | nd | xxx | xxx | xxx | xxx | xxx | xxx | xxx | xxx | xxx |
| 18 | | 100 | — | — | nd | nd | nd | / | / | / | xxx | xxx | xxx | xxx | xxx | xxx |
| 19 | | 50 | — | — | nd | nd | nd | / | / | / | / | / | / | xxx | xxx | xxx |
| 20 | | 10 | — | — | nd | nd | nd | / | / | / | / | / | / | xxx | xxx | xxx |
| 21 | | 0 | — | — | nd | nd | nd | / | / | / | / | / | / | / | / | / |
| 22 | CAL- | 250 | 0.005 | — | nd | nd | nd | / | / | / | / | / | / | / | / | / |
| 23 | 15 | 250 | — | 0.05 | nd | nd | nd | / | / | / | / | / | / | / | / | / |
| 24 | | 250 | — | — | nd | nd | nd | xxx | xxx | xxx | xxx | xxx | xxx | xxx | xxx | xxx |
| 25 | | 100 | — | — | nd | nd | nd | / | / | / | / | / | / | xxx | xxx | xxx |
| 26 | | 50 | — | — | nd | nd | nd | xxx | / | / | / | / | / | xxx | xxx | xxx |
| 27 | | 10 | — | — | nd | nd | nd | / | / | / | / | / | / | / | / | / |
| 28 | | 0 | — | — | nd | nd | nd | / | / | / | / | / | / | / | / | / |
| 29 | non- | 250 | — | — | nd | nd | nd | / | / | / | / | / | / | / | / | / |
| 30 | degraded | 100 | — | — | nd | nd | nd | / | / | / | / | / | / | / | / | / |
| 31 | PS20 | 50 | — | — | nd | nd | nd | / | / | / | / | / | / | / | / | / |
| 32 | | 10 | — | — | nd | nd | nd | / | / | / | / | / | / | / | / | / |
| 33 | | 0 | — | — | nd | nd | nd | / | / | / | / | / | / | / | / | / |
| 34 | MML-60* | 0 | — | — | nd | nd | nd | xxx | xxx | xxx | xxx | xxx | xxx | xxx | xxx | xxx |

Results

The solubility limit of lauric and myristic acid was assessed by spiking of FFA stock solutions into histidine acetate buffer pH 5.5 to attain target concentration: of 0-30 μg/mL for lauric acid and 0-12 μg/mL myristic acid (see Example 1). Samples were incubated at 2-8° C. and were inspected for visible particles after 0, 7, 14 and 28 days using the Seidenader (Table 3A) and E/P box (Table 31B). Formation of many visible particles (>10 in Seidenader and >7 in E/P box) was observed already after 1 day in samples containing at least 20/8 μg/mL lauric/myristic acid, whereas lower concentrations of FFAs resulted in an overall lower number of particles and a delayed particle onset. Since visual inspection with the Seidenader machine is performed at 1.5-fold magnification the overall number of particles per container was higher as compared to the E/P box analysis. The solubility limit was defined as the concentration of lauric and myristic acid above which many visible particles (>10 for Seidenader and >7 for E/P box) were observed after 28 days for all three vials of the triplicate set. As shown in Table 3, in samples containing at least 12.5 μg/mL lauric acid and 5 μg/mL myristic acid, the concentration of free fatty acids exceeded the solubility limit and FFAs crushed out as visible particles. In conclusion, all samples with lower lauric and myristic concentrations were considered to be below the solubility limit.

Subsequently, FFA stock solutions were spiked into aqueous buffered solutions (20 mM histidine acetate buffer pH 5.5) containing different amounts of aluminum ranging from 0 to 250 ppb (Example 2). Thereby, the final concentration of fatty acids in the sample was below the solubility limit (10/4, 7.5/3, 5/2, 0/0 μg/mL lauric/myristic acid) as previously determined with the exception of one sample containing 25/10 μg/mL lauric/myristic acid which was used as a positive control. Samples containing the highest content of aluminum (250 ppb) were additionally spiked with EDTA at 0.005% (w/v). All samples were incubated at 2-8° C. for up to 28 days and inspected for visible particles using the Seidenader (Table 4A) and E/P box (Table 4B). As shown in Table 4, the presence of aluminum led to the formation of FFA particles even below the FFA solubility limit. The extent of visible particle formation as well as the onset were dependent on the aluminum concentration and surprisingly also inversely dependent on the FFA concentration. Samples containing the highest concentration of aluminum displayed the earliest particle onset as well as the highest number of particles. This effect was even more pronounced for samples containing decreasing amounts of FFAs. As demonstrated by the Seidenader results, even trace amounts of aluminum of 10 ppb were sufficient to complex free fatty acids and form visible particles. Samples containing only fatty acids (below the solubility limit) but no aluminum or vice versa did not form significant numbers of visible particles (≤10 in Seidenader and ≤7 in E/P box) upon incubation at 2-8° C. for up to 28 days.

Formation of visible FFA particles could be suppressed by the addition of 0.005% (w/v) EDTA to samples containing the highest aluminum concentrations (250 ppb) and FFA levels below the solubility limit.

Turning to the more specific problem of FFA release from polysorbates in aqueous (parenteral) protein or antibody formulations, it has previously been disclosed that Hydrolytic degradation of polysorbate is mainly driven by the presence of host-cell proteins (HCPs) which are co-purified with the active pharmaceutical ingredient (API) and can catalyze hydrolysis of the ester bond in polysorbate. (Labrenz 2014; Dixit et al. 2016). It is already known that different enzymes possess different specificities towards mono- or poly-ester components of polysorbate, leading to different polysorbate degradation patterns (McShan et al. 2016).

In Example 3, polysorbate was artificially degraded by two different enzymes which were previously immobilized onto beads to allow for a precise control over the degradation level. MML preferentially degrades higher-order esters, whereas CAL uniformly degrades mono- as well as higher oder esters (Graf et al. 2020).

The solubility limit of partially hydrolyzed polysorbate degraded with either MML or CAL was determined by spiking of PS20 stock solutions into histidine acetate buffer pH 5.5 to a total concentration of 0.4 mg/mL. Samples were incubated at 2-8° C. and visually inspected (F/P box) for the presence of visible particles for up to 28 days. Formation of many visible particles (>7 in E/P box) was immediately observed after spiking of polysorbate degraded by MML at 40% and 60%. In contrast, no visible particles were observed for CAL samples at the initial time point (d0), even at the highest degradation level (60%). After incubation at 2-8° C., visible particles were observed already at 20% or 30% polysorbate degradation for MML and CAL samples, respectively.

The solubility limit for each polysorbate degradation series was defined as the critical degradation degree above which many visible particles (>7 for EIP box) were observed after 28 days for all three vials of the triplicate set.

Samples with lower polysorbate degradation degrees (<20% for MML and <30% for CAL) were defined to be below the solubility limit.

In a next step, partially degraded PS20 solutions were spiked into aqueous buffered solutions (20 mM histidine acetate buffer pH 5.5) containing different amounts of aluminum ranging from 0 to 250 ppb (Example 4). Thereby, the final concentration of polysorbate degradants was below the solubility limit (10% and 15%) as previously determined with the exception of one sample containing polysorbate degraded with MML at 40% which was used as a positive control. Samples containing the highest content of aluminum (250 ppb) were additionally spiked with either DTPA at 0.05 mM or EDTA at 0.005% (w/v). All samples were incubated at 2-8° C. for up to 28 days and visually inspected (F/P box). As shown in Table 6, the presence of aluminum led to the formation of FFA particles even below critical polysorbate degradation levels. Again both, the extent of visible particle formation as well as the particles onset were dependent on the aluminum concentration and surprisingly also inversely dependent on the PS20 degradation degree and the degradation pattern.

Samples containing the highest concentration of aluminum displayed the earliest particle onset as well as the highest number of particles. This effect was slightly more pronounced for lower polysorbate degradation degrees (10%). Interestingly, when aluminum was present, CAL samples showed visible particle formation even at very low levels of aluminum (10 ppb) and significant earlier (after 7 days) as compared to the corresponding MML samples.

No visible particles formed in samples spiked with undegraded polysorbate or containing no aluminum upon incubation at 2-8° C. for up to 28 days.

The formation of visible FFA particles was thus successfully prevented by the addition of 0.05 mM DTPA or 0.005% (w/v) EDTA to samples containing the highest aluminum concentrations (250 ppb) and partially degraded polysorbate below the solubility limit.

### Example 5: Particle Formation in a Pertuzumab Formulation with and without Chelators

Pertuzumab (mAb1) was formulated at 30 mg/mL in 20 mM histidine acetate buffer pH 6.0, 120 mM sucrose, supplemented with 0.2 mg/mL HP PS20 or pure oleic acid (POA) PS80, 0 or 10 mM methionine and 0 or 0.05 mM chelator (DTPA or EDTA) as summarized in Table 7. The formulated drug product was filled into 20 cc borosilicate vials (14.0 mL) and stored at 2-8° C. 30 vials of each formulation were prepared.

TABLE 7
Sample composition of different mAb1 formulations

| | | | | | | | Format |
|---|---|---|---|---|---|---|---|
| | mAb | Histidine | Sucrose | PS20/80 | Methionine | | and fill |
| Formulation | [mg/mL] | acetate | [mM] | [mg/mL] | [mM] | Chelator | volume |
| −01 | 30 | 20 mM | 120 | 0.2 | — | — | 14.0 |
| −02 | mg/mL | pH 6.0 | | HP PS20 | 10 | — | mL |
| −03 | | | | | 10 | 0.05 mM | in |
| | | | | | | DTPA | 20 cc |
| | | | | | | | vial |
| −04 | | | | | 10 | 0.05 mM | |
| | | | | | | EDTA | |
| −05 | | | | 0.2 | 10 | 0.05 mM | |
| | | | | POA | | DTPA | |
| | | | | PS80 | | | |

Visual inspection (Seidenader or E/P) was performed on 30 vials per formulation after 6 months of incubation at 2-8° C. Vials were either inspected within 1 hour after removal from 2-8° C. storage (cold sample solution) or after equilibration to ambient temperature for 4 hours.

Results:

Table 8 and 9 summarize the visual inspection results using E/P method or Sidenader, respectively, after 6 months storage at 2-8° C. The overall particle count of the cold sample solution was overall higher compared to the samples after equilibration to ambient temperature which is an indication that particles mainly consist of free fatty acids or fatty acid salts which have lower solubility at lower temperatures. Comparing the particles in different formulations after equilibration, the number of containers with particles was quite similar for all five formulations (1-3 containers out of 30). The total number of particles and was

TABLE 8
Visual inspection (EP) after 6 months (2-8° C.)

| | Container with | Total number of | Max. |
|---|---|---|---|
| | particles | particles | number of |
| Formulation | (≥1) | (30 container) | particles |

Cold sample solution

| | | | |
|---|---|---|---|
| −01 | 2 | 4 | 2 |
| −02 | 6 | 10 | 4 |
| −03 | 3 | 3 | 1 |
| −04 | 4 | 4 | 1 |
| −05 | 0 | 0 | 0 |

Equilibrated sample solution (room temperature)

| | | | |
|---|---|---|---|
| −01 | 1 | 4 | 4 |
| −02 | 3 | 3 | 1 |
| −03 | 1 | 1 | 1 |
| −04 | 2 | 2 | 1 |
| −05 | 2 | 5 | 4 |

TABLE 9
Enhanced visual inspection (Seidenader) after 6 months (2-8° C.)

| | Container with | Total number of | Max. |
|---|---|---|---|
| | particles | particles | number of |
| Formulation | (≥1) | (30 container) | particles |

Cold sample solution

| | | | |
|---|---|---|---|
| −01 | 30 | 163 | >10 |
| −02 | 29 | 190 | >10 |
| −03 | 29 | 186 | >10 |
| −04 | 28 | 89 | >10 |
| −05 | 21 | 81 | >10 |

Equilibrated sample solution (room temperature)

| | | | |
|---|---|---|---|
| −01 | 18 | 76 | >10 |
| −02 | 16 | 46 | >10 |
| −03 | 14 | 36 | >10 |
| −04 | 4 | 12 | 6 |
| −05 | 10 | 18 | 4 |

### Example 6: Spiking Study with Different PS20 and PS80 Grades and DTPA

### Methods

Preparation of Enzymatically Degraded PS20 and PS80

Super refined (SR) PS20 and PS80, high purity (HP) PS20 and PS80, as well as pure oleic acid (POA) PS80 were enzymatically hydrolyzed by 10% using immobilized enzymes (Graf et al. 2020) (mucor miehei lipase (MML), Candida antarctica lipase (CAL) and Candida antarctica lipase B (CALB)). These enzymes possess different specificities towards mono- and higher-order esters, whereas MML mainly degrades higher-order ester species in PS, CAL targets mono- and higher order esters and CALB preferentially degrades mono-esters.

Spiking Studies with Partially Degraded Polysorbates (PS20 and PS80) and Aluminum

Diluted aluminum solutions comprising in 20 mM histidine acetate pH 5.5 were prepared as described above and filled into 20 mL type I borosilicate glass vials (Schott, Mainz, DE). Samples containing either 0 or 250 ppb of aluminum (Al³) were spiked with different PS stock solutions (HP PS20, SR PS20, HP PS80, SR PS80, POA PS80; degradation level of 0 or 10%). In addition, samples containing 250 ppb aluminum were supplemented with 0.05 mM diethylenetriaminepentaacetic acid (DTPA) prior spiking with partially degraded PS. Vials were sealed with 20 mm teflonized injection stoppers (D777-1, Daikyo) and aluminum crimp caps and homogenized on a MaxQ™ 4000 Benchtop Orbital Shaker (Thermo Scientific™, Waltham, Mass., USA) for 1 hour at 25° C.

Samples prepared with partially degraded PS20 or PS80 resulted in FFA concentrations below the solubility limit. Different grades of non-degraded polysorbate 20 and 80 served as negative controls. All samples were prepared in triplicates.

Samples were stored at 5° C. and particle formation was assessed for up to 22 days by visual inspection using a black/white panel according Ph. Eur. 2.9.20. and enhanced visual inspection (Seidenader V 90-T instrument) as described above.

Results

Different grades of PS20 (SR, HP) and PS80 (SR, HP, POA) were partially degraded by 10% using different enzymes which possess different substrate specificities. PS stock solutions (0 or 10% degradation) were spiked into aqueous buffer solutions containing no (0 ppb) or 250 ppb of aluminum, or 250 ppb aluminum and additionally 50 μM DTPA. Vials were incubated at 2-8° C. and the formation of visible particles was monitored by visual inspection (E/P) and enhanced visual inspection (Seidenader). As shown in Table 10 (visible particles by E/P), in samples containing 10% degraded PS20 or PS80 but no aluminum, no visible particles were observed during the course of the study. Many visible particles formed in samples containing partially degraded PS20 and aluminum, independent of the enzyme used for degradation, whereas for samples containing partially degraded PS80 and aluminum, significantly fewer or no visible particles were observed. For all grades of PS no particles were observed in presence of the chelator (DTPA). Interestingly, the non-degraded HP PS20 and HP PS80 controls containing 250 ppb aluminum formed visible particles, whereas all other controls were particle free. This observation might be explained by the fact that HP grade PS contains significantly higher levels of free fatty acids in the raw material compared to the corresponding SR grades (Doshi et al. 2020a). Although these levels are far below the FFA solubility levels they might be high enough for FFA metal nucleation.

TABLE 10
Visible particle formation (E/P) after spiking of PS stock solutions
(degradation level of 0 or 10%) into aqueous buffered solutions (20 mM histidine
acetate buffer pH 5.5) containing either no or 250 ppb of aluminum (n = 3). Samples
containing 250 ppb aluminum were additionally formulated with a chelator (DTPA).
Samples were analyzed for the presence of visible particles after storage at 2-8° C. for
0 to 22 days using the E/P box. The cumulative content of particles per container (20
mL) was classified as ‘many particles (>7, xxx)’, ‘few particles (5-7, xx)’, or
‘practically free of particles (0-4, /)’. Sample containing either non-degraded PS no
salt were used as negative controls. Results are reported as the average number of
particles in 3 containers, d = day of inspection, CALB = candida antarctica lipase B,
MML = mucor miehei lipase, CAL = candida antarctica lipase, DTPA =
diethylenetriaminepentaacetic acid.

| Polysorbate | | | Aluminum | Visible Particles (E/P) | | | | |
|---|---|---|---|---|---|---|---|---|
| Grade | Degradation | Enzyme | conc. (ppb) | d1 | d8 | d15 | d18 | d22 |
| HP | 10% | CALB | 0 | / | / | / | / | / |
| PS20 | | MML | | / | / | / | / | / |
| | | CAL | | / | / | / | / | / |
| | | CALB | 250 | / | / | / | xxx | xxx |
| | | MML | | xx | xxx | xxx | xxx | xxx |
| | | CAL | | xxx | xxx | xxx | xxx | xxx |
| | | CALB | 250 + | / | / | / | / | / |
| | | MML | 50 μM | / | / | / | / | / |
| | | CAL | DTPA | / | / | / | / | / |
| | 0% | — | 250 | / | xxx | xx | xxx | xxx |
| SR | 10% | CALB | 0 | / | / | / | / | / |
| PS20 | | MML | | / | / | / | / | / |
| | | CAL | | / | / | / | / | / |
| | | CALB | 250 | xxx | xxx | xxx | xx | xxx |
| | | MML | | xxx | xxx | xxx | xxx | xxx |
| | | CAL | | xxx | xxx | xxx | xxx | xxx |
| | | CALB | 250 + | / | / | / | / | / |
| | | MML | 50 μM | / | / | / | / | / |
| | | CAL | DTPA | / | / | / | / | / |
| | 0% | — | | / | / | / | / | / |
| HP | 10% | CALB | 0 | / | / | / | / | / |
| PS80 | | MML | | / | / | / | / | / |
| | | CAL | | / | / | / | / | / |
| | | CALB | 250 | xx | / | / | / | / |
| | | MML | | / | / | / | / | / |
| | | CAL | | / | / | / | xxx | xxx |
| | | CALB | 250 + | / | / | / | / | / |
| | | MML | 50 μM | / | / | / | / | / |
| | | CAL | DTPA | / | / | / | / | / |
| | 0% | — | 250 | / | xx | / | xx | xx |
| SR | 10% | CALB | 0 | / | / | / | / | / |
| PS80 | | MML | | / | / | / | / | / |
| | | CAL | | / | / | / | / | / |
| | | CALB | 250 | / | / | / | / | / |
| | | MML | | / | xx | / | / | xx |
| | | CAL | | / | / | / | xx | / |
| | | CALB | 250 + | / | / | / | / | / |
| | | MML | 50 μM | / | / | / | / | / |
| | | CAL | DTPA | / | / | / | / | / |
| | 0% | — | 250 | / | / | / | / | / |
| POA | 10% | CALB | 0 | / | / | / | / | / |
| PS80 | | MML | | / | / | / | / | / |
| | | CAL | | / | / | / | / | / |
| | | CALB | 250 | / | / | / | / | / |
| | | MML | | xx | xx | xxx | xx | xxx |
| | | CAL | | xx | xx | xx | xx | xx |
| | | CALB | 250 + | / | / | / | / | / |
| | | MML | 50 μM | / | / | / | / | / |
| | | CAL | DTPA | / | / | / | / | / |
| | 0% | — | 250 | / | / | / | / | / |

Table 11 shows the corresponding enhanced visual inspection results for HP PS2, SR PS20, HP PS80, SR PS80 and POA PS80. Controls comprising partially degraded PS in absence of aluminum stayed free or essentially free of particles, whereas all samples containing partially degraded PS and aluminum instantaneously formed many visible particles. Particle formation was significantly mitigated in presence of DTPA, independent on the grade of PS or the enzyme used for degradation. Again, in samples comprising non-degraded HP PS2r and HP PS8d and aluminum many particles were formed, whereas fewer or no particles w (observed in the other non-degraded controls (with aluminum) were observed.

TABLE 11
Visible particle formation (Seidenader) after spiking of PS stock solutions
(degradation level of 0 or 10%) into aqueous buffered solutions (20 mM histidine acetate
buffer pH 5.5) containing either no or 250 ppb of aluminum (n = 3). Samples containing 250
ppb aluminum were additionally formulated with a chelator (DTPA). Samples were analyzed
for the presence of visible particles after storage at 2-8° C. for 0 to 22 days using the
Seidenader instrument. The cumulative content of particles per container was classified as
′many particles (>10, xxx)′, ′few particles (6-10, xx)′, ′essentially free of particles (1-5, x)′, or
′free of particles (0, /)′. Sample containing either non-degraded PS or no salt were used as
negative controls. d = day of inspection, CALB = candida antarctica lipase B, MML =
mucor miehei lipase, CAL = candida antarctica, DTPA =
diethylenetriaminepentaacetic acid

| Polysorbate | | | Aluminum | Visible Particles (Seidenader) | | | | |
|---|---|---|---|---|---|---|---|---|
| Grade | Degradation | Enzyme | conc. (ppb) | d1 | d8 | d15 | d18 | d22 |
| HP PS20 | 10% | CALB | 0 | x | / | x | x | x |
| | | MML | | / | x | x | x | x |
| | | CAL | | x | x | x | x | x |
| | | CALB | 250 | xx | xxx | xxx | xxx | xxx |
| | | MML | | xxx | xxx | xxx | xxx | xxx |
| | | CAL | | xxx | xxx | xxx | xxx | xxx |
| | | CALB | 250 + | xx | x | x | xx | xxx |
| | | MML | DTPA | x | x | x | x | / |
| | | CAL | | xx | x | x | xx | xx |
| | 0% | — | 250 | xx | xxx | xxx | xxx | xxx |
| SR PS20 | 10% | CALB | 0 | x | x | / | x | x |
| | | MML | | / | x | / | x | xx |
| | | CAL | | x | / | x | x | x |
| | | CALB | 250 | xxx | xxx | xxx | xxx | xxx |
| | | MML | | xxx | xxx | xxx | xxx | xxx |
| | | CAL | | xxx | xxx | xxx | xxx | xxx |
| | | CALB | 250 + | x | / | / | x | x |
| | | MML | DTPA | x | / | / | x | x |
| | | CAL | | / | x | x | x | x |
| | 0% | — | | x | / | / | x | xx |
| HP PS80 | 10% | CALB | 0 | x | x | / | x | x |
| | | MML | | x | / | x | x | x |
| | | CAL | | / | / | / | / | / |
| | | CALB | 250 | xx | xxx | xxx | xxx | xxx |
| | | MML | | xxx | xxx | xxx | xxx | xxx |
| | | CAL | | xx | xxx | xxx | xxx | xxx |
| | | CALB | 250 + | / | / | x | / | xx |
| | | MML | DTPA | x | x | x | x | x |
| | | CAL | | x | x | x | xx | x |
| | 0% | — | 250 | xx | xxx | xxx | xxx | xxx |
| SR PS80 | 10% | CALB | 0 | x | / | x | / | x |
| | | MML | | x | / | x | x | x |
| | | CAL | | / | / | / | x | / |
| | | CALB | 250 | xxx | xxx | xxx | xxx | xxx |
| | | MML | | xxx | xxx | xxx | xxx | xxx |
| | | CAL | | xxx | xxx | xxx | xxx | xxx |
| | | CALB | 250 + | x | / | x | x | x |
| | | MML | DTPA | x | x | x | / | x |
| | | CAL | | x | x | x | x | x |
| | 0% | — | 250 | / | / | / | / | x |
| POA | 10% | CALB | 0 | x | x | / | xx | xx |
| PS80 | | MML | | x | x | x | x | x |
| | | CAL | | x | x | / | x | x |
| | | CALB | 250 | xx | xx | xxx | xxx | xxx |
| | | MML | | xx | xxx | xxx | xxx | xxx |
| | | CAL | | xxx | xxx | xxx | xxx | xxx |
| | | CALB | 250 + | x | / | x | x | x |
| | | MML | DTPA | x | x | x | x | xx |
| | | CAL | | x | x | x | xx | xx |
| | 0% | — | 250 | x | x | x | xxx | xx |

### Example 7: Screening of Metal Salts and Chelators

### Methods

FFA Particle Nucleation by Dynamic Light Scattering (DLS)

DLS experiments were performed on DynaPro(R) plate reader (Wyatt, Santa Barbara, Calif.). The DLS plate reader is flushed with nitrogen 5 hours before commencing the measurements and cooled down to 5° C. during the entire duration of the measurements. 200 μL of sample solutions comprising of 20 μg lauric acid (LA) in 20 mM L-Histidine buffer at pH 6.0 supplemented with 6% v/v DMSO, and varying amounts of metal ions (Al³⁺, Fe³, Zn²⁺, Mg²⁺, Ca²⁺, Ni²⁺) were mixed in the cuvettes of black glass bottom 96 well plates (Greiner Bio-One GmbH, Frickenhausen, Germany). FFA nucleation and particle growth were measured over 40-70 hours using a 633 nm laser and a backscatter detection system at 158°. The hydrodynamic particle radius (rH) was determined by fitting a cumulant fit to the obtained auto-correlation function. The lower and upper boundary for the cumulant fit was set to lag times τ of 10 μs and 1000 μs respectively. The laser power was adjusted before each measurement sequence and kept constant during the entire measurement. The attenuation level was set to zero for all measurements in order to collect the maximum amount of scattered light.

Impact of Aluminum (Al) Concentration on Particle Kinetics

Al sample solutions (100×) in 20 mM Histidine buffer pH 6.0 were prepared from a sterile 50 ppm Al stock solution (20 mM Glycine pH 2.5) and were spiked into the DLS assay buffer at target concentrations of 0, 20, 40, 60 and 100 ppb Al. Particle nucleation and growth was measured over a period of 40 hours as described above.

Quantitative analysis was performed by analyzing the intensity of the scattered laser light of the FFA particles over time by fitting a sigmoidal Boltzmann function to the growth curves.

$$f ( x ) = \frac{A - B}{1 + {e}^{( x - {x}_{0} ) / dx}} + B \qquad ( Eq . 1 )$$

where A denotes to the initial and B to the final value, x₀ is the center or inflection point of the sigmoidal curve and dx is the time constant.

Interaction of Bi- and Trivalent Cations with Lauric Acid

Aluminium (Al), Iron (Fe), Zink (Zn), Magnesium (Mg), Calcium (Ca) and Nickel (Ni) stock solutions were prepared at 4 mM in Milli-Q water pH 2.5 from their respective salts and stored at 2-8° C. until used. Diluted sample solutions (100×) were freshly prepared in Milli-Q water pH 2.5. The DLS assay buffer was added to the salt sample solutions to attain metal ion target concentrations of 0, 1, 3, 10 and 30 μM. Particles size (rH) and intensity were measured over a period of 70 h as described above.

FFA Metal Nucleation in Presence of Chelators

Diethylenetriaminepentaacetic acid (DTPA), Ethylenediaminetetraacetic acid (EDTA), 1,3-Diaminopropane-N,N,N′,N′-tetraacetic acid (PDTA) and Tetrasodium N,N-Bis(carboxymethyl)-L-glutamate (GLDA) stock solutions were prepared in 20 mM Histidine buffer pH 5.5 and diluted to the desired concentration in DLS assay buffer. For the Al nucleation study, a metal ion target concentration of 2 μM was used. The final concentration of DTPA, EDTA, GLDA and PDTA was either 0, 0.5, 1.0, 1.5, 2 and 20 μM, resulting in chelator to Al molar ratios of 0, 0.25, 0.5, 0.75, 1.0 and 10, respectively. For the Fe nucleation study, the target concentration of Fe was 4 μM. The final concentration of DTPA was either 0, 0.04, 0.4, 2.0, 4.0 or 40 μM resulting in molar ratios (DTPA:Fe) of 0, 0.01, 0.1, 0.5, 1.0 and 10, respectively. DLS measurements were conducted over 50 hours at 5° C. as described above.

DTPA Prevents FFA Metal Nucleation in Presence of Real Glass Leachables

Representative glass leachables solutions were prepared according to a procedure disclosed in Allmendinger et al. (Allmendinger et al. 2021). In brief, 6 mL of glycine solution at pH 10 were filled into 6 mL Fiolax® vials (Schott AG, Müllheim, Germany, and Schott North America Inc., NY, USA), stoppered with D777-1 serum stoppers (DAIKYO Seiko Ltd., Tokyo, Japan) and subjected to one autoclaving cycle (121° C., 20 min). The glass leachable content in the sample after dilution was 37 ppb Al, 43 ppb B, 430 ppb Si, and 0 ppb of Na, K Ca, corresponding to 1.4 μM of Al, 4.0 μM of B and 15.3 μM of Si. The DTPA concentration was either 0, 0.04, 0.4, 2.0, 4.0 or 40 μM resulting in molar ratios of 0, 0.03, 0.3, 1.5, 2.9 and 29 (DTPA:Al), or 0. 0.002, 0.02, 0.1, 0.2 and 1.9 (DTPA to glass leachables), respectively. DLS measurements were conducted over 50 hours at 5° C. as described above.

### Results

Interaction of Metal Cations with Lauric Acid

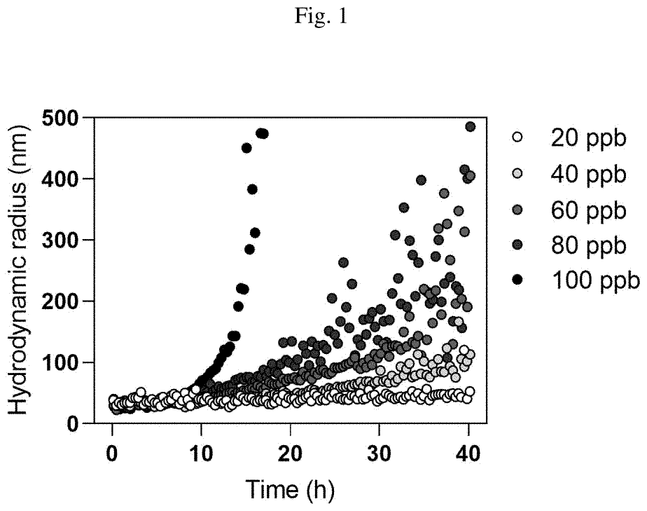

To assess the risk of other bi- and trivalent metal impurities which could potentially be introduced during DP manufacturing or storage, a surrogate assay based on DLS was established. DLS can be used to capture particle formation and growth in the size range of 0.3 nm to 10 μm (Panchal et al. 2014) and therefore was suitable to detect the early nucleation event (FFA-metal interaction). Since proteins and polysorbate micelles would interfere with the assay, measurements were conducted in aqueous solutions containing lauric acid (below the solubility limit), which is the main degradation product from hydrolytic PS20, and DMSO (6% v/v) to increase the LA solubility. In a first experiment, different Al concentrations were used to trigger FFA complexation and subsequent particle formation. As shown in FIG. 1, nanoparticles were formed in all formulations containing Al, whereas no particles were observed for the controls without either Al or FFA (data not shown). The particle size of Al-containing samples increased over time, whereby the increase in size scaled with higher Al concentrations.

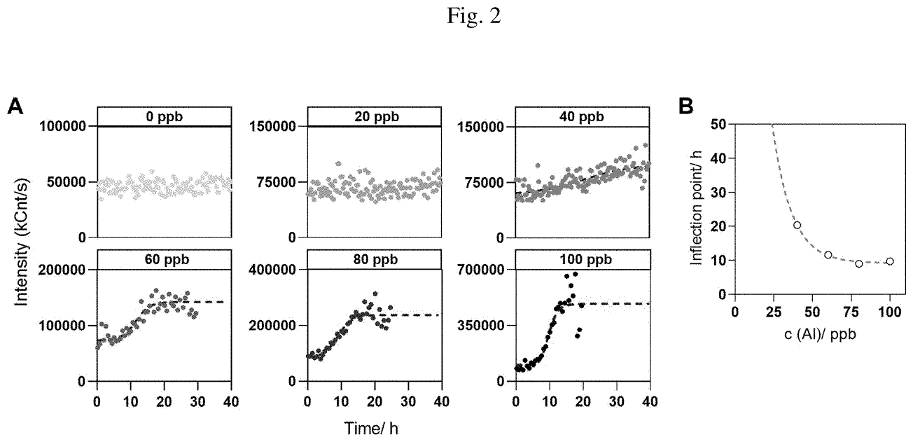

As shown in FIG. 2B, the scattering intensity of samples containing Al was initially higher compared to the control samples without al (5000 kCnt/s). For the sample with 40 ppb Al a slight increase in intensity was observed over time, whereas samples with 60-100 ppb Al showed a sharp increase in intensity, which could be fitted with a sigmoidal curve to determine the inflection points (FIG. 2B). Since the intensity range and consequently the slope of the sigmoidal fit is significantly increased for samples containing high Al levels, the tₒₙₛₑₜ could not be used to assess the particle growth kinetics (data not shown). Instead, the inflection point of the sigmoidal curve was used since it is independent of the intensity range.

As shown in FIG. 2B the inflection point of the sigmoidal curve fit exponentially decayed with increasing Al concentrations indicating that the event of complexation and particle growth can be significantly accelerated with trace levels of Al (40-60 ppb), whereas a further increase in Al (beyond 80 ppb) did not result in a further shift of the inflection point. These results suggest that although larger particles are formed in presence of higher Al concentrations the particle growth kinetics is not affected above a concentration of 80 ppb. For very low concentrations (20 ppb) the inflection point was calculated to be around 65 hours and was therefore not captured in this experiment.

In a second study, different bi- (Ca, Mg, Zn, Ni) and trivalent (Al, Fe) cations were screened for their propensity to interact with lauric acid and form FFA salt particles.

For a better comparison, equal molar amounts of metal ions ranging from 0-30 μM were used. A translation into respective ppb concentrations is provided in Table 12.

TABLE 12
Conversion of metal ion concentration.

| | Concentration used in DLS experiment | | | | |
|---|---|---|---|---|---|
| | 0 μM | 1 μM | 3 μM | 10 μM | 30 μM |
| Metal ion | Corresponding metal ion concentration in ppb | | | | |
| Al³⁺ | 0 | 27 | 81 | 270 | 809 |
| Fe³⁺ | 0 | 56 | 168 | 558 | 1675 |
| Ca²⁺ | 0 | 40 | 120 | 401 | 1202 |
| Mg²⁺ | 0 | 24 | 73 | 243 | 729 |
| Zn²⁺ | 0 | 65 | 196 | 654 | 1961 |
| Ni²⁺ | 0 | 59 | 176 | 587 | 1761 |

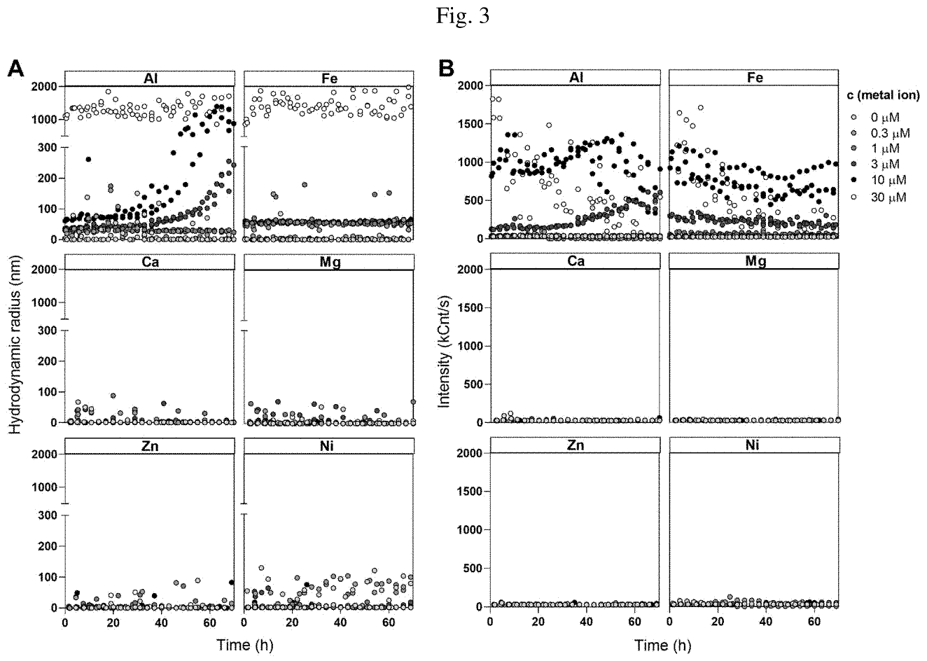

The change in particle size (r_{H}) and intensity as a function of metal cation type and concentration was assessed over a period of 70 hours. As shown in FIG. 3 particle formation and growth was only observed for trivalent metal ions (Fe, Al), whereas the presence of divalent cations (Ca, Mg, Ni, Zn) did not lead to FFA salt particle formation or growth. Interestingly, Al and Fe behaved quite differently. While an increase in Al concentration (0-10 μM) resulted in the formation of larger particles and faster particle growth, the addition of equal amounts of Fe resulted in instant particle formation (r_{H} of 50-60 nm) but no further increase in particle size over time. However, the scattering intensity increased with increasing levels of Al and Fe, suggesting that although FFA-Fe particles do not grow over time, the number of particles is still increasing.

For both trivalent cations, the highest concentration (30 μM) led to the formation of very large particles. As the particle size was close to the upper limit of detection for DLS, no further increase in size could be observed. In addition, these large particles seemed to sediment over time, as indicated by a gradual decrease in scattering intensity (FIG. 3B).

Based on these results it can be concluded that the presence of divalent cations poses a low risk for FFA salt particle formation in the investigated concentration range, whereas trivalent cations, such as Al³⁺ and Fe³⁺, can interact with negatively charged FFAs, even at very low concentrations.

Protective Effect of Chelators Against FFA Metal Nucleation

To assess the protective effect of chelators, DTPA, EDTA, GLDA and PDTA were spiked into solutions containing lauric acid and 4 uM Al at different concentrations, ranging from 0-40 μM which corresponds to a molar ratio (chelator to Al) of 0-10.

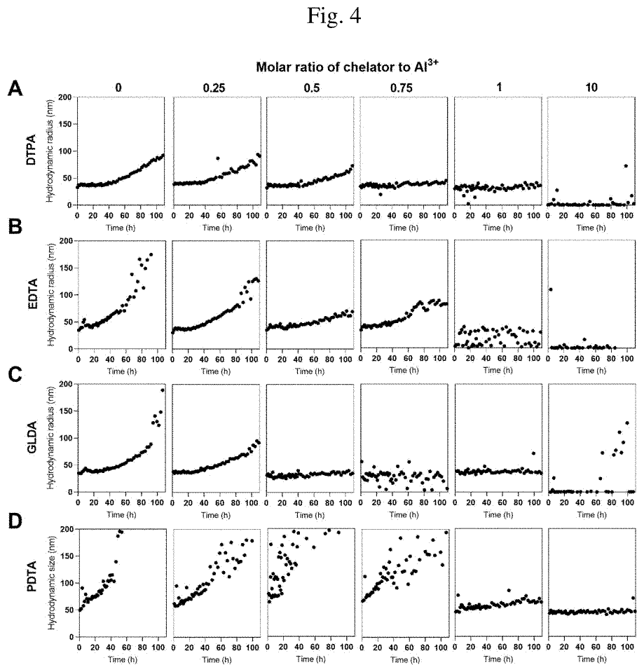

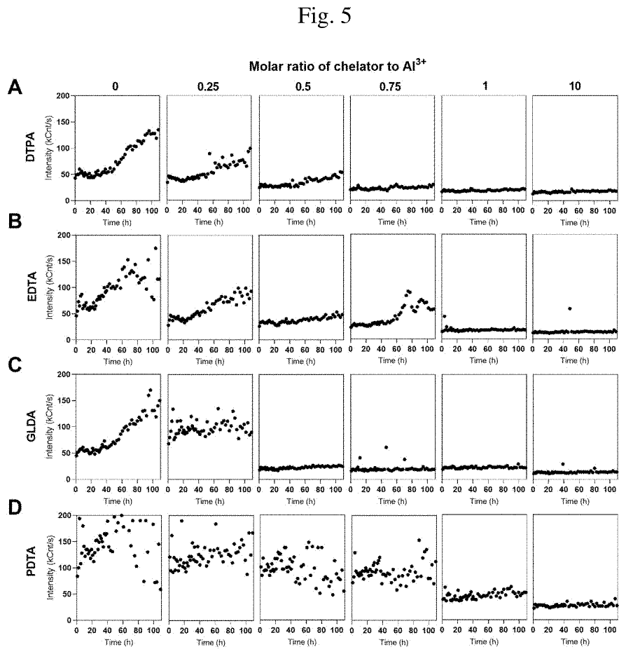

As shown in FIG. 4 and FIG. 5, an increase in chelator concentration resulted in an overall decrease in scattering intensity and particle size, as well as a slower particle growth over time. At a molar ratio of at least 1:1 (chelator to Al), particle formation and growth was efficiently suppressed for all chelators. Slight differences in efficiency between chelators can be attributed to the chemical structure. While EDTA, GLDA and PDTA are tetra-acetic acid which can complex multivalent ions as a six-toothed chelating agent, DTPA is a pentetic acid with eight coordinate bond forming sites (five carboxylate oxygen atoms and three nitrogen atoms). The carboxylate donor groups become increasingly protonated when reducing the pH (Eivazihollagh et al. 2017), resulting in less charged chelator species and therefore, weaker metal complexation. As DTPA has more donor atoms than EDTA, GLDA and PDTA it can efficiently complex multivalent cations at slightly acidic pH.

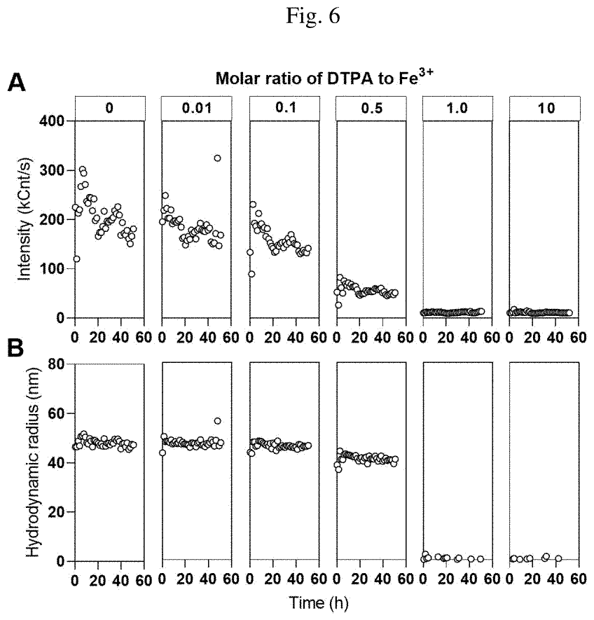

The protective effect of DTPA was also assessed in presence of lauric acid and 4 μM Fe (FIG. 6). In contrast to Al, FFA metal nucleation with Fe leads to instant nanoparticle formation in the DLS assay but no significant particle growth over time. Addition of equimolar amounts of DTPA resulted in complete suppression of particle formation and growth, similarly as for Al. At a DTPA to Fe ratio of 0.5, the scattering intensity was significantly decreased but the particle radius was only slightly changed, suggesting that primarily the number of particles was reduced.

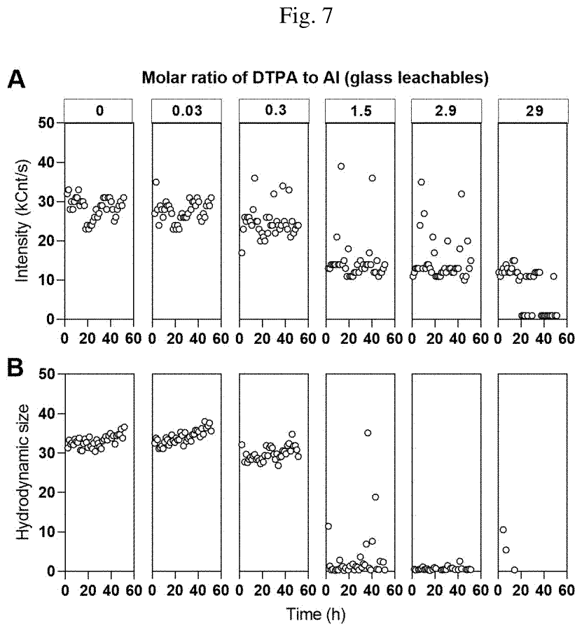

The addition of DTPA to samples comprising of lauric acid and real glass leachables extracted from Exp51 borosilicate glass vials was also efficient in mitigating particle formation at molar ratios of at least 1.5 of chelator to Al (FIG. 7).

### REFERENCES

Allmendinger, Andrea; Lebouc, Vanessa; Bonati, Lucia; Woehr, Anne; Kishore, Ravuri S. K.; Abstiens, Kathrin (2021): Glass Leachables as a Nucleation Factor for Free Fatty Acid Particle Formation in Biopharmaceutical Formulations. In: Journal of pharmaceutical sciences 110 (2), S. 785-795. DOI: 10.1016/j.xphs.2020.09.050.
Ditter, Dominique; Mahler, Hanns-Christian; Gohlke, Linus; Nieto, Alejandra; Roehl, Holger; Huwyler, Joerg et al. (2018): Impact of Vial Washing and Depyrogenation on Surface Properties and Delamination Risk of Glass Vials. In: Pharmaceutical research 35 (7), S. 146. DOI: 10.1007/s11095-018-2421-6.
Dixit, Nitin; Salamat-Miller, Nazila; Salinas, Paul A.; Taylor, Katherine D.; Basu, Sujit K. (2016): Residual Host Cell Protein Promotes Polysorbate 20 Degradation in a Sulfatase Drug Product Leading to Free Fatty Acid Particles. In: Journal of pharmaceutical sciences 105 (5), S. 1657-1666. DOI: 10.1016/j.xphs.2016.02.029.
Doessegger, Lucette; Mahler, Hanns-Christian; Szczesny, Piotr; Rockstroh, Helmut; Kallmeyer, Georg; Langenkamp, Anja et al. (2012): The potential clinical relevance of visible particles in parenteral drugs. In: Journal of pharmaceutical sciences 101 (8), S. 2635-2644. DOI: 10.1002/jps.23217.
Doshi, Nidhi; Demeule, BarthWlemy; Yadav, Sandeep (2015): Understanding Particle Formation: Solubility of Free Fatty Acids as Polysorbate 20 Degradation Byproducts in Therapeutic Monoclonal Antibody Formulations. In: Molecular pharmaceutics 12 (11), S. 3792-3804. DOI: 10.1021/acs.molpharmaceut.5b00310.
Doshi, Nidhi; Fish, Raphael; Padilla, Karina; Yadav, Sandeep (2020a): Evaluation of Super Refined™ Polysorbate 20 With Respect to Polysorbate Degradation, Particle Formation and Protein Stability. In: Journal of pharmaceutical sciences 109 (10), S. 2986-2995. DOI: 10.1016/j.xphs.2020.06.030.
Doshi, Nidhi; Giddings, Jamie; Luis, Lin; Wu, Arthur, Ritchie, Kyle; Liu, Wenqiang et al. (2021): A Comprehensive Assessment of All-Oleate Polysorbate 80: Free Fatty Acid Particle Formation, Interfacial Protection and Oxidative Degradation. In: Pharmaceutical research 38 (3), S. 531-548. DOI: 10.1007/s11095-021-03021-z.
Doshi, Nidhi; Martin, Joelle; Tomlinson, Anthony (2020b): Improving Prediction of Free Fatty Acid Particle Formation in Biopharmaceutical Drug Products: Incorporating Ester Distribution during Polysorbate 20 Degradation. In: Molecular pharmaceutics 17 (11), S. 4354-4363. DOI: 10.1021/acs.molpharmaceut.0c00794.
Doyle Drbohlav, Laura M.; Sharma, Anant Navanithan; Gopalrathnam, Ganapathy; Huang, Lihua; Bradley, Scott (2019): A Mechanistic Understanding of Polysorbate 80 Oxidation in Histidine and Citrate Buffer Systems-Part 2. In: PDA journal of pharmaceutical science and technology 73 (4), S. 320-330. DOI: 10.5731/pdajpst.2018.009639.
Eivazihollagh, Alireza; Bäckström, Joakim; Norgren, Magnus; Edlund, Håkan (2017): Influences of the operational variables on electrochemical treatment of chelated Cu(II) in alkaline solutions using a membrane cell. In: Journal of Chemical Technology & Biotechnology) 92 (6), S. 1436-1445. DOI: 10.1002/jctb.5141.
European Directorate for the Quality of Medicines: Histidine Monography 01/2017:0911. In: European Pharmacopeia 10.3 2021.
European Directorate for the Quality of Medicines: Particulate contamination: Visible particles [2.9.20]. In: European Pharmacopeia 10.0 2020.
Glücklich, Nils; Dwivedi, Mridula; Carle, Stefan; Buske, Julia; Mader, Karsten; Garidel, Patrick (2020): An in-depth examination of fatty acid solubility limits in biotherapeutic protein formulations containing polysorbate 20 and polysorbate 80. In: International journal of pharmaceutics 591, S. 119934. DOI: 10.1016/j.ijpharm.2020.119934.
Gopalrathnam, Ganapathy; Sharma, Anant Navanithan; Dodd, Steven Witt; Huang, Lihua (2018): Impact of Stainless Steel Exposure on the Oxidation of Polysorbate 80 in Histidine Placebo and Active Monoclonal Antibody Formulation. In: PDA journal of pharmaceutical science and technology 72 (2), S. 163-175. DOI: 10.5731/pdajpst.2017.008284.
Graf, Tobias; Abstiens, Kathrin; Wedekind, Frank; Elger, Carsten; Haindl, Markus; Wurth, Christine; Leiss, Michael (2020): Controlled polysorbate 20 hydrolysis—A new approach to assess the impact of polysorbate 20 degradation on biopharmaceutical product quality in shortened time. In: European journal of pharmaceutics and biopharmaceutics 152, S. 318-326. DOI: 10.1016/j.ejpb.2020.05.017.
Hall, Troii; Sandefur, Stephanie L.; Frye, Christopher C.; Tuley, Tammy L.; Huang, Lihua (2016): Polysorbates 20 and 80 Degradation by Group XV Lysosomal Phospholipase A2 Isomer X1 in Monoclonal Antibody Formulations. In: Journal of pharmaceutical sciences 105 (5), S. 1633-1642. DOI: 10.1016/j.xphs.2016.02.022.
Hewitt, Daniel; Zhang, Taylor; Kao, Yung-Hsiang (2008): Quantitation of polysorbate 20 in protein solutions using mixed-mode chromatography and evaporative light scattering detection. In: Journal of chromatography. A 1215 (1-2), S. 156-160. DOI: 10.1016/j.chroma.2008.11.017.
Honemann, Maximilian N.; Wendler, Jan; Graf, Tobias; Bathke, Anja; Bell, Christian H. (2019): Monitoring polysorbate hydrolysis in biopharmaceuticals using a QC-ready free fatty acid quantification method. In: Journal of chromatography. B, Analytical technologies in the biomedical and life sciences 1116, S. 1-8. DOI: 10.1016/j.jchromb.2019.03.030.
Kishore, Ravuri S. K.; Kiese, Sylvia; Fischer, Stefan; Pappenberger, Astrid; Grauschopf, Ulla; Mahler, Hanns-Christian (2011a): The degradation of polysorbates 20 and 80 and its potential impact on the stability of biotherapeutics. In: Pharmaceutical research 28 (5), S. 1194-1210. DOI: 10.1007/s11095-011-0385-x.
Kishore, Ravuri S. K.; Pappenberger, Astrid; Dauphin, Isabelle Bauer; Ross, Alfred; Buergi, Beatrice; Staempfli, Andreas; Mahler, Hanns-Christian (201 lb): Degradation of polysorbates 20 and 80: studies on thermal autoxidation and hydrolysis. In: Journal of pharmaceutical sciences 100 (2), S. 721-731. DOI: 10.1002/jps.22290.
Kranz, Wendelin; Wuchner, Klaus; Corradini, Eleonora; Berger, Michelle; Hawe, Andrea (2019): Factors Influencing Polysorbate's Sensitivity Against Enzymatic Hydrolysis and Oxidative Degradation. In: Journal of pharmaceutical sciences 108 (6), S. 2022-2032. DOI: 10.1016/j.xphs.2019.01.006.
Labrenz, Steven R. (2014): Ester hydrolysis of polysorbate 80 in mAb drug product: evidence in support of the hypothesized risk after the observation of visible particulate in mAb formulations. In: Journal of pharmaceutical sciences 103 (8), S. 2268-2277. DOI: 10.1002/jps.24054.
Lippold, Steffen; Koshari, Stijn H. S.; Kopf, Robert; Schuller, Rudolf; Buckel, Thomas; Zarraga, Isidro E.; Koehn, Henning (2017): Impact of mono- and poly-ester fractions on polysorbate quantitation using mixed-mode HPLC-CAD/ELSD and the fluorescence micelle assay. In: Journal of pharmaceutical and biomedical analysis 132, S. 24-34. DOI: 10.1016/j.jpba.2016.09.033.
McShan, Andrew C.; Kei, Pervina; Ji, Junyan A.; Kim, Daniel C.; Wang, Y. John (2016): Hydrolysis of Polysorbate 20 and 80 by a Range of Carboxylester Hydrolases. In: PDA journal of pharmaceutical science and technology 70 (4), S. 332-345. DOI: 10.5731/pdajpst.2015.005942.
Panchal, Jainik; Kotarek, Joseph; Marszal, Ewa; Topp, Elizabeth M. (2014): Analyzing subvisible particles in protein drug products: a comparison of dynamic light scattering (DLS) and resonant mass measurement (RMM). In: The AAPS journal 16 (3), S. 440-451. DOI: 10.1208/s12248-014-9579-6.
Yarbrough, Melissa; Hodge, Tamara; Menard, Daniele; Jerome, Richard; Ryczek, Jeff; Moore, David et al. (2019): Edetate Disodium as a Polysorbate Degradation and Monoclonal Antibody Oxidation Stabilizer. In: Journal of pharmaceutical sciences 108 (4), S. 1631-1635. DOI: 10.1016/j.xphs.2018.11.031.
Zhou, Shuxia; Schöneich, Christian; Singh, Satish K. (2011): Biologics formulation factors affecting metal leachables from stainless steel. In: AAPS PharmSciTech 12 (1), S. 411-421. DOI: 10.1208/s12249-011-9592-3.

1. A stable aqueous composition comprising a protein, one or more pharmaceutically acceptable excipients and at least one chelator.

2. The composition according to claim 1, wherein said chelator is selected from the group consisting of Ethylenediaminetetraacetic acid (EDTA), Diethylenetriaminepentaacetic acid (DTPA), Ethyleneglycol-bis(β-aminoethyl)-N,N,N′,N′-tetraacetic Acid (EGTA), N-Carboxymethyl-N′-(2-hydroxyethyl)-N,N′-ethylenediglycine (HEDTA), ethylenediamine-N,N′-bis(2-dihydroxyphenylacetic acid) (EDDHA), 1,3-Diaminopropane-N,N,N′,N′-tetraacetic acid (PDTA), Tetrasodium N,N-Bis(carboxymethyl)-L-glutamate (GLDA), citrate, malonate, tartrate, ascorbate, salicylic acid, aspartic acid, and glutamic acid.

3. The composition according to claim 2, wherein the chelator is Ethylenediaminetetraacetic acid (EDTA) or Diethylenetriaminepentaacetic acid (DTPA).

4. The composition according to claim 1, wherein said chelator is present in a concentration from 0.0005 to 2.0% (w/v).

5. The composition according to any one of claim 1, wherein said protein is an antibody or a monoclonal antibody.

6. A method of making stable parenteral protein or stable parenteral antibody preparations or of preventing formation of visible particles in parenteral protein or antibody preparations, the method comprising adding at least one chelator to a parenteral protein or parenteral antibody composition.

7. The method of claim 6, wherein said parenteral protein or antibody preparations remain free of visible particles for the entire time of their authorized shelf life.

8. (canceled)

9. The method according to claim 6, wherein the chelators are selected from the group consisting of Ethyleneglycol-bis(β-aminoethyl)-N,N,N′,N′-tetraacetic Acid (EGTA), N-Carboxymethyl-N′-(2-hydroxyethyl)-N,N′-ethylenediglycine (HEDTA), ethylenediamine-N,N′-bis(2-dihydroxyphenylacetic acid) (EDDHA), 1,3-Diaminopropane-N,N,N′,N′-tetraacetic acid (PDTA), Tetrasodium N,N-Bis(carboxymethyl)-L-glutamate (GLDA), citrate, malonate, tartrate, ascorbate, salicylic acid, aspartic acid, glutamic acid, Ethylenediaminetetraacetic acid (EDTA), and Diethylenetriaminepentaacetic acid (DTPA or Pentetic Acid), and are present in concentrations ranging from 0.0005 to 2.0%.

10. The method according to claim 9, wherein the chelator is EDTA or DTPA.

11. The method of claim 7, wherein said visible particles comprise at least one multivalent cation and free fatty acids cleaved from surfactants present in the parenteral protein preparation.

12. A pharmaceutical dosage form comprising a composition according to claim 1, in a container.

13. (canceled)

14. The method of claim 7, wherein the surfactants include one or both of polysorbate 20 and polysorbate 80.

15. A pharmaceutical dosage form comprising a preparation obtained according to the method of claim 6, in a container.
